(19) United States
(12) Patent Application Publication
(10) Pub. No.: US 2010/0138957 A1
(43) Pub. Date: Jun. 3, 2010

(54) HYBRID PEPTIDES HAVING ANTIMICROBIAL ACTIVITY AND METHODS OF MAKING AND USING HYBRID PEPTIDES

(76) Inventors: Zhijian T. Li, Altamonte Springs, FL (US); Dennis J Gray, Howey-in-the-Hills, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/524,541

(22) PCT Filed: Feb. 1, 2008
(86) PCT No.: PCT/US08/01353

§ 371 Date: Jan. 6, 2010

Related U.S. Application Data
(60) Provisional application No. 60/887,636, filed on Feb. 1, 2007.

(51) Int. Cl. A01H 1/00; C07K 14/00; C07H 21/04; C12N 5/10; A01P 15/00; A01H 5/00; A61K 38/16; A61K 31/7088

(52) U.S. Cl. 800/279; 530/324; 536/23.1; 435/419; 800/298; 514/12; 514/44 R

(57) ABSTRACT

The present invention concerns the development and utilization of hybrid lytic peptides derived from non-venomous molecular sources to confer a high level of sustainable resistance to phytopathogens in transgenic plants. In an exemplified embodiment, a composition of the invention comprises a cecropin-pleurocidin hybrid peptide of 27 amino acids. The peptide was designed based on optimization of critical molecular and physiochemical parameters. Peptides of the invention offer significantly enhanced antimicrobial activity and molecular properties associated with low cytotoxicity. Transgenic plants of grapevine (Vitis vinifera) that express a peptide of the invention show antimicrobial activity against xylem-limited phytopathogenic bacterium Xylella fastidiasa at a level significantly higher than that from other existing lytic peptides. Thus, the hybrid peptides of the invention can be utilized as an antimicrobial agent for agricultural use.

### CROSS REFERENCE TO RELATED APPLICATION

This application claims the benefit of U.S. Provisional Application Ser. No. 60/887,636, filed Feb. 1, 2007, which is hereby incorporated by reference herein in its entirety, including any figures, tables, nucleic acid sequences, amino acid sequences, and drawings.

### BACKGROUND OF THE INVENTION

### Natural AMPs

Antimicrobial peptides (AMPs) are small molecules with lytic activity that are produced by numerous organisms including, but not necessarily limited to, bacteria, plants, vertebrates and invertebrates. These peptides are crucial components of the hosts' innate immune system in the defense against invading microorganisms. Since the isolation of cecropins from pupae of the silkmoth (Hyalophora cecropia) in the early 1980's (Steiner et al., 1981), more than 880 AMPs have been documented (Brogden, 2005). These peptide molecules and related genes are being extensively studied in order to understand the mechanisms underlying their antimicrobial and host defense activities.

Antimicrobial peptides from different organisms display a diversity of sequence and structural characteristics and biological functions. Naturally occurring AMPs are 12 to 50 amino acids-long and capable of forming various secondary structures including α-helices, β-sheets, or both, extended helices, and loops. Most AMPs are polycationic with a net positive charge and contain both hydrophobic and hydrophilic domains, making them amphipathic or soluble in both water and membrane environments (Boman, 2003). Antimicrobial peptides have a relatively high differential binding affinity to negatively charged phospholipids, which are the major constituents of bacterial and viral membranes. Hence, AMPs tend to inset into the hydrophobic interior of the membranes and cause conformational changes, leading to membrane permeabilization/destabilization, leakage of cellular electrolytes and ultimately cell death (Sitaram and Nagaraj, 1999; Zhang et al., 2001). All reported AMPs exhibit a broad spectrum of activity against various microbial targets, including Gram-negative and \ Gram-positive bacteria, fungi and enveloped viruses (Hancock and Lehrer, 1998). In addition to their ability to induce membrane pores and the leakage of cytoplasmic contents, it was also suggested that AMPs also exert their inhibitory activity by blocking the biosynthesis of macromolecules including DNA, RNA, and/or protein, eventually resulting in the death of target cells (Friedrich et al., 2000; Patrzykat et al., 2002).

Several models have been proposed to elucidate the mode of lytic action associated with various AMPs (see Brogden, 2005 for the latest review). These models emphasize the way in which AMP molecules orientate/align themselves in the membrane environment while forming transmembrane pores. In the barrel stave model, AMP molecules align themselves perpendicular to the membrane and form a bundle, much like staves of a barrel (Ehrenstein and Lecar, 1977). In the carpet model, AMPs accumulate and orientate themselves parallel to the membrane surface in a carpet-like manner. Membrane disintegration and cell death occur when AMP molecules interact with the anionic phospholipid head groups in a detergent-like manner, leading to the formation of micelles and disruptive conformational changes in the membranes (Pouny and Shai, 1992; Gazit et al., 1995). Finally, in the toroidal pore model AMPs orientate themselves perpendicular to the membrane—similar to the barrel stave model except that AMP molecules insert themselves into the membrane forming toroidal channels with phospholipid monolayers bending continuously through the pore (Matsuzaki et al., 1996). It should be noted that in all these models the formation of α-helical structures is crucial for AMPs to confer membrane spanning activity.

In the last two decades, there has been substantial interest in the utilization of naturally occurring AMPs to protect plants from both bacterial and fungal pathogens using transgenic technologies. However, the direct use of genes encoding several natural AMPs such as cecropins proved relatively ineffective and failed to confer significant level of resistance to phytopathogens in transgenic plants (Hightower et al., 1994; Florack et al., 1995; Hancock and Lehrer, 1998).

### Existing Synthetic Hybrid AMPS

Over the last two decades, concerted efforts have been made to modify peptide amino acid sequence and develop synthetic hybrid lytic peptides in order to improve the molecular stability and antimicrobial activity of natural AMPs. Unfortunately, transgenic plants expressing these modified AMPs, analogues or hybrids showed limited resistance to phytopathogens (Boman et al., 1989; Huang and McBeath, 1997; Owens and Heutte, 1997; Arce et al., 1999; Norelli et al. 1999; Osusky et al., 2000; Scorza and Gray, 2001 U.S. Pat. No. 6,232,528).

Among the synthetic AMPs tested thus far, several cecropin-melittin hybrids have produced promising results and have attracted the attention of scientists in search of novel sources of plant disease resistance.

Cecropins were first discovered in the hemolyph of pupae of the silkmoth (Hyalophora cecropia) (Steiner et al., 1981), but have since been found in numerous insect species and even in member of the animal kingdom (Boman and Hultmark, 1987; Lee et al., 1989). Structural analyses have indicated that cecropins from insects are usually composed of 35-39 amino acid residues with two different helical domains connected by a flexible non-helical hinge region. Those from animals, such as cecropin P1 from pig, form amphiphilic α-helix over nearly the whole length of the molecule (Gazit et al., 1995). By using fluorescent labeling technique, Gazit et al., (1995) demonstrated that monomers of cecropins are capable of binding to the acidic membrane surface and disrupting the lipid packing in the bilayers via the carpet-like mechanism. As a result, the integrity of bacterial membrane is destroyed and bacterial cells die. Cecropins have potent lytic activity against a wide variety of Gram-positive and Gram-negative bacteria, with no known adverse activity against eukaryotic cells.

The N-terminal domain (head region) of cecropins contain a relatively high content of basic amino acid residues and folds into a perfect amphipathic α-helix, while the C-terminal domain (tail region) is rich in hydrophobic residues and forms a more hydrophobic helix (Van Hofsten et al., 1985). The positively charged N-terminal amphipathic α-helix can easily span a negatively charged bacterial lipid membrane and exhibit voltage-dependent ion-permeable pore-forming properties (Christensen et al., 1988). Accordingly, this N-terminal domain (residues 1 to 13) has been recognized as a fusion partner candidate for the construction of hybrid AMPs.

Melittin is a 26-residue peptide that is a major toxic component in the venom of the European honey bee (Apis mellifera) (Habermann, 1972). Melittin also has a helix-hinge-helix structure similar to that of cecropins, but with opposite polarity, i.e., a hydrophobic N terminus and an amphipathic C terminus. Extensive structure-function studies revealed that the amphiphilic helical N-terminal segment (residues 1-14) of melittin possesses channel-forming capabilities and thus is responsible for most of the antibacterial activity. The hinge region plays a crucial role in modulating the hemolytic activity. The C-terminal segment (residues 20 to 26) of melittin had no effect on lytic activity (Sitaram and Nagaraj, 1999). The formation of transmembrane pores induced by melittin was found to be consistent with the toroidal pore model (Yang et al., 2001). In spite of their exceptionally high antimicrobial activity, the equally potent hemolytic and allergenic activities of melittin have precluded its practical use as a means to confer disease resistance in transgenic plants.

In an effort to develop synthetic AMPs with enhanced bacterial membrane lysing capabilities, but with reduced hemolytic activity, hybrid peptides composed of various segments of cecropins and melittin were synthesized and tested (Boman et al., 1989). These studies revealed that chimeric peptides containing the amphiphilic 1-13 or 1-8 N-terminal segment of cecropin A and the 1-13 or 1-18 regions of melittin showed a broad-spectrum of antimicrobial activity up to 100-fold higher than the activity of natural cecropin A alone. These hybrid peptides had relatively lower hemolytic activity and did not lyse sheep red blood cells at 50-200 times higher concentrations as compared to melittin (Boman et al., 1989 and Wade et al., 1990).

Wade et al., (1990) had first created a hybrid AMP called CEME (KWKLFKKIGIGAVLKVLTTGLPALIS) (SEQ ID NO: 3) by combining cecropin 1-8 plus melittin 1-18 residues. Later, Piers et al. (1994) noted the presence of multiple positively charged residues (KRKR) in the C-terminus of melittin, and incorporated an additional 4-residue sequence extension with two positively charged residues (KLTK) to the C terminus of CEME to produce a modified hybrid AMP called CEMA (KWKLFKKIGIGAVLKV TTGLPALKTLK) (SEQ ID NO: 4). These researchers demonstrated that CEMA had an improved binding affinity to and favorable interactions with lipid membranes leading to greater membrane-permeabilizing capability (Piers et al., 1994; Friedrich et al., 1999). Details regarding the design and use of CEMA and related peptides can be found in several recent US patents by Hancock et al. (U.S. Pat. Nos. 5,593,866; 5,707,855; 6,288,212 and 6,818,407). Recently, Osusky et al. (2000) incorporated a 6-residue (MALEHM) (SEQ ID NO: 5) extension at the N terminus of CEMA to generate a variant hybrid AMP termed MsrA1 (MALEHMKWKLFKKIGIGAVLKVLTTGLPALKTLK) (SEQ ID NO: 6) in an attempt to presumably dampen excessive lytic activity, and demonstrated that the expression of the MsrA1 gene in transgenic potato plants resulted in improved broad-spectrum resistance to bacterial and fungal phytopathogens.

It should be pointed out that in spite of the observed disease resistance, health safety issues concerning the incorporation of these hybrid AMPs derived from venomous molecular sources, such as melittin, in human food crops have not been assuaged.

### Pleurocidin

Pleurocidin was first identified from the skin secretions or mucus of the epithelial layer of winter flounder (Pseudopleuronectes americanus) (Cole et al., 1997). It is a 25-residue cationic peptide capable of forming a rigid α-helical structure in a membrane environment. In addition, pleurocidin is heat-stable, salt-tolerant and insensitive to physiological concentrations of magnesium and calcium (Cole et al., 2000). In vitro tests showed that pleurocidin possesses a moderate broad-spectrum of antimicrobial activity against a large number of Gram-positive and Gram-negative bacteria and fungi. These unique molecular and biological properties suggest that pleurocidin is one of the major components in the host's innate immune system playing an important role in the first line of mucosal defense for the flatfish against pathogenic microorganisms in hostile environments (Cole et al., 2000; Syvitski et al., 2005).

It should be noted that in vitro tests revealed that the level of antimicrobial activity of pleurocidin remained moderate as compared to other strong lytic AMPs. For instance, 28 to 62 μg per ml of pleurocidin is required to achieve a MIC against Pseudomonas aeruginosa, while 2.8 μg per ml of CEMA is sufficient to reach a MIC against the same pathogen (Cole et al., 2000; Piers et al., 1994).

Nevertheless, pleurocidin is one of the safest natural AMPs identified thus far. It has been proposed that this peptide be used as an antimicrobial agent in food applications. Every day, millions of people worldwide are affected by microorganism-induced food borne illnesses. Due to the ever-increasing reluctance to use harmful chemicals in human foods, numerous natural AMPs have been tested over the years to serve as replacements for chemical preservatives and antibiotics now used for food preservation. Up to today, nisin, a bacteriocin isolated from lactic acid bacteria, is the only natural AMP approved by the FDA as a commercial food preservative, even though it has been shown that this peptide has a limited spectrum of antimicrobial activity, lacks the ability to kill Gram-negative bacteria and fungi and functions only at low pH (Hancock and Lehrer, 1998). Recently, Burrowes et al. (2004) investigated both the antimicrobial and cytotoxic activities of pleurocidin. Their findings demonstrated that pleurocidin, unlike nisin, has excellent broad-spectrum antimicrobial activity. It was effective against 17 of the 18 bacterial and fungal microorganisms tested, including several Gram-negative pathogenic bacteria, such as Vibrio parahaemolyticus that thrives in estuarine and marine environments and is responsible for major outbreaks of food borne illnesses due to the use of contaminated seafood products. Noticeably, the capability of pleurocidin to kill a variety of pathogenic microorganisms may also be attributable to its ability to enter target cells and block the synthesis of macromolecules, including DNA and protein, at sublethal concentrations (Patrzykat et al., 2002).

Pleurocidin has minimal hemolytic activity and no cytotoxic effects on human intestinal epithelial cells (Burrowes et al., 2004). Studies using intraperitoneal injections of AMPs into juvenile coho salmon revealed that pleurocidin was more effective against lethal vibriosis caused by pathogenic Vibrio bacteria, but had a significantly lower mortality rate when compared to CEME (Jia et al., 2000). Pleurocidin-like analogues modified to incorporate an N-terminal lysine cap or 4- to 7-residue at N-terminal substitutions were also found to be capable of conferring various levels of antimicrobial activity (U.S. Pat. No. 6,288,212; U.S. Pat. No. 6,818,407). Results of all these studies suggest that pleurocidin is an ideal molecular candidate for the construction of active hybrid AMPs that can be used as a source of disease resistance in plants and animals.

Xylella fastidiosa is an insect transmitted bacterium that only resides in xylem vessels of the plant (i.e., xylem-limited). Xylem vessels are responsible for the transportation of water and dissolved ions. Water and ions typically enter the plant through its root system and then move upwards and are distributed through a network of xylem vessels. Various strains of this bacterium are the causal agent of several diseases including Pierce's disease (PD) of grapevine (Vitis vinifera), phony peach disease (PPD), plum leaf scald, citrus variegated chlorosis (CVC), and leaf scorch of almond, coffee, elm, oak, oleander pear, and sycamore. All cultivars of V. vinifera, which produce the majority of table and wine grapes, are susceptible to PD. Susceptible grape vines eventually succumb to PD due to severe water stress as movement of water and nutrients through the xylem are stopped as a consequence of the aggregation of X. fastidiosa. Diseases caused by X. fastidiosa are most prevalent in the southeastern United States. At the present time, PD is the single most important factor limiting grape production in this region. The recent increase PD in the west coast grape-growing areas caused by the introduction of an efficient leafhopper vector of X. fastidiosa, the glassy-winged sharpshooter (Homalodisca coagulata), has posed a significant adventive threat to the California grape industry. Presumably, a transgenic plant that produced an antimicrobial peptide in xylem sap would retard growth of X. fastidiosa and, thus, be resistant to PD.

### BRIEF SUMMARY OF THE INVENTION

The present invention concerns the development and utilization of hybrid lytic peptides derived from non-venomous molecular sources to confer a high level of sustainable resistance to phytopathogens in transgenic plants. In an exemplified embodiment, a composition of the invention comprises a cecropin-pleurocidin hybrid peptide of 27 amino acids. The peptide was designed based on optimization of critical molecular and physiochemical parameters. The invention also comprises the design and utilization of a hybrid peptide of the invention having antimicrobial activity. Peptides of the invention offer significantly enhanced antimicrobial activity and molecular properties associated with low cytotoxicity. Transgenic plants of grapevine (Vitis vinifera) that express a peptide of the invention show antimicrobial activity against xylem-limited phytopathogenic bacterium Xylella fastidiosa at a level significantly higher than that from other existing lytic peptides. Thus, the hybrid peptides of the invention can be utilized as an antimicrobial agent for agricultural use.

### BRIEF DESCRIPTION OF THE DRAWINGS

FIGS. 1A-1C show a comparison of conformational parameter profiles for α-helix. FIG. 1A (B-passerin); FIG. 1B (Pleurocidin); and FIG. 1C (MsrA1). Bar values represent secondary structure propensity for α-helical conformation for each amino acid residue of specified peptide predicted by a sliding window calculation of the cumulative index for three successive residues, using previously reported scales (Deleage and Roux, 1987) and Vector NTI DNA/protein analysis software.

FIGS. 2A-2C show a comparison of hydrophobicity indices determined by HPLC. FIG. 2A (B-passerin); FIG. 2B (Pleurocidin); and FIG. 2C (MsrA1). Bar values of hydrophobicity represent the standardized retention times of amino acid residues on reversed-phase high-performance liquid chromatography at pH 3.0 and pH 7.5 previously determined by Cowan and Whittaker (1990), and were rendered by using Vector NTI DNA/protein analysis software. A positive index value indicates increasing hydrophilicity, whereas a negative value suggests increasing hydrophobicity.

FIGS. 3A-3D show analysis of unstructured regions of AMP peptides using Deleage/Roux definition. Graph for each compared peptide was obtained from GlobPlot after the input of specific peptide sequence. FIG. 3A (B-passerin); FIG. 3B (MsrA1); FIG. 3C (Pleurocidin); and FIG. 3D (CEME). Regions of significant disorder were identified and marked by the GlobPlot software (http://globplot.embl.de).

FIG. 4 shows physical map of transformation vector containing the B-passerin gene. Vector pBPS was constructed based on a pBIN-19 binary plasmid. 35S-3′, cauliflower mosaic virus (CaMV) 35S RNA transcript termination and polyadenylation signal sequence; TMV, 5′-leader (Omega) sequence of tobacco mosaic virus (TMV); dCaMV 35S, doubly enhanced CaMV 35S promoter; dCsVMV, doubly enhanced cassaya vein mosaic virus promoter; AMV, 5′-leader sequence of alfalfa mosaic virus; RB and LB, right and left border sequences of T-DNA region; EGFP, enhanced green fluorescent protein gene; NPTII, neomycin phosphotransferase gene; Kan III, bacterial kanamycin resistance gene.

FIG. 5 shows PD symptom development 5 months after bacterial inoculation. Plants were grown in the greenhouse for 1 to 2 month prior to inoculation with pathogenic Xylella fastidiosa. After inoculation, plants were maintained using standard procedures. Representative plants were displayed. CK^{R}, local tolerant control variety Blanc du Bois; CK^{s}, susceptible non-transformed Thompson Seedless; B-passerin-expressing plants, independent lines of pBPS-transformed Thompson Seedless.

FIG. 6 shows PD resistance performance of transgenic grape plants expressing the B-passerin gene. A scale for PD resistance performance was created by using numbers 0 to 5, i.e., a plant that died from PD disease received a score of 0, whereas score numbers 1 to 5 were given to plants showing progressively lessened severity of PD symptoms and increasing plant vigor. A symptomless plant with robust PD resistance acquired a score of 5. Bar values represent average indices of at least 10 independent plant lines from 5 repeated inoculation experiments. Standard errors for average values were indicated.

### BRIEF DESCRIPTION OF THE SEQUENCES

SEQ ID NO: 1 is a polynucleotide sequence encoding a B-passerin peptide of the invention.

SEQ ID NO: 2 is an amino acid sequence of a B-passerin peptide of the invention.

SEQ ID NO: 3 is the amino acid sequence of a hybrid AMP designated as CEME.

SEQ ID NO: 4 is the amino acid sequence of a hybrid AMP designated as CEMA.

SEQ ID NO: 5 is the amino acid sequence of a peptide extension to the CEMA peptide.

SEQ ID NO: 6 is the amino acid sequence of a hybrid AMP designated as MsrA1.

SEQ ID NO: 7 is a polynucleotide sequence encoding a B-passerin peptide of the invention.

SEQ ID NO: 8 is an amino terminal deletion of the amino acid sequence shown in SEQ ID NO: 2.

SEQ ID NO: 9 is an amino terminal deletion of the amino acid sequence shown in SEQ ID NO: 2.

SEQ ID NO: 10 is an amino terminal deletion of the amino acid sequence shown in SEQ ID NO: 2.

SEQ ID NO: 11 is an amino terminal deletion of the amino acid sequence shown in SEQ ID NO: 2.

SEQ ID NO: 12 is an amino terminal deletion of the amino acid sequence shown in SEQ ID NO: 2.

SEQ ID NO: 13 is a carboxy terminal deletion of the amino acid sequence shown in SEQ ID NO: 2.

SEQ ID NO: 14 is a carboxy terminal deletion of the amino acid sequence shown in SEQ ID NO: 2.

SEQ ID NO: 15 is a carboxy terminal deletion of the amino acid sequence shown in SEQ ID NO: 2.

SEQ ID NO: 16 is a carboxy terminal deletion of the amino acid sequence shown in SEQ ID NO: 2.

SEQ ID NO: 17 is a carboxy terminal deletion of the amino acid sequence shown in SEQ ID NO: 2.

SEQ ID NO: 18 is an amino and carboxy terminal deletion of the amino acid sequence shown in SEQ ID NO: 2.

SEQ ID NO: 19 is an amino acid sequence of a B-passerin peptide of the invention that does not include terminal amino acid extensions (MA and TK).

SEQ ID NO: 20 is an amino and carboxy terminal deletion of the amino acid sequence shown in SEQ ID NO: 2.

SEQ ID NO: 21 is an amino and carboxy terminal deletion of the amino acid sequence shown in SEQ ID NO: 2.

SEQ ID NO: 22 is an amino and carboxy terminal deletion of the amino acid sequence shown in SEQ ID NO: 2.

SEQ ID NO: 23 is an amino and carboxy terminal deletion of the amino acid sequence shown in SEQ ID NO: 2.

SEQ ID NO: 24 is an amino terminal addition of the amino acid sequence shown in SEQ ID NO: 2.

SEQ ID NO: 25 is an amino terminal addition of the amino acid sequence shown in SEQ ID NO: 2.

SEQ ID NO: 26 is an amino terminal addition of the amino acid sequence shown in SEQ ID NO: 2.

SEQ ID NO: 27 is an amino terminal addition of the amino acid sequence shown in SEQ ID NO: 2.

SEQ ID NO: 28 is an amino terminal addition of the amino acid sequence shown in SEQ ID NO: 2.

SEQ ID NO: 29 is a carboxy terminal addition of the amino acid sequence shown in SEQ ID NO: 2.

SEQ ID NO: 30 is a carboxy terminal addition of the amino acid sequence shown in SEQ ID NO: 2.

SEQ ID NO: 31 is a carboxy terminal addition of the amino acid sequence shown in SEQ ID NO: 2.

SEQ ID NO: 32 is a carboxy terminal addition of the amino acid sequence shown in SEQ ID NO: 2.

SEQ ID NO: 33 is a carboxy terminal addition of the amino acid sequence shown in SEQ ID NO: 2.

SEQ ID NO: 34 is an amino and carboxy terminal addition of the amino acid sequence shown in SEQ ID NO: 2.

SEQ ID NO: 35 is an amino and carboxy terminal addition of the amino acid sequence shown in SEQ ID NO: 2.

SEQ ID NO: 36 is an amino and carboxy terminal addition of the amino acid sequence shown in SEQ ID NO: 2.

SEQ ID NO: 37 is an amino and carboxy terminal addition of the amino acid sequence shown in SEQ ID NO: 2.

SEQ ID NO: 38 is an amino and carboxy terminal addition of the amino acid sequence shown in SEQ ID NO: 2.

SEQ ID NO: 39 is an amino terminal addition of the amino acid sequence shown in SEQ ID NO: 19.

SEQ ID NO: 40 is an amino terminal addition of the amino acid sequence shown in SEQ ID NO: 19.

SEQ ID NO: 41 is an amino terminal addition of the amino acid sequence shown in SEQ ID NO: 19.

SEQ ID NO: 42 is an amino terminal addition of the amino acid sequence shown in SEQ ID NO: 19.

SEQ ID NO: 43 is an amino terminal addition of the amino acid sequence shown in SEQ ID NO: 19.

SEQ ID NO: 44 is a carboxy terminal addition of the amino acid sequence shown in SEQ ID NO: 19.

SEQ ID NO: 45 is a carboxy terminal addition of the amino acid sequence shown in SEQ ID NO: 19.

SEQ ID NO: 46 is a carboxy terminal addition of the amino acid sequence shown in SEQ ID NO: 19.

SEQ ID NO: 47 is a carboxy terminal addition of the amino acid sequence shown in SEQ ID NO: 19.

SEQ ID NO: 48 is a carboxy terminal addition of the amino acid sequence shown in SEQ ID NO: 19.

SEQ ID NO: 49 is an amino and carboxy terminal addition of the amino acid sequence shown in SEQ ID NO: 19.

SEQ ID NO: 50 is an amino and carboxy terminal addition of the amino acid sequence shown in SEQ ID NO: 19.

SEQ ID NO: 51 is an amino and carboxy terminal addition of the amino acid sequence shown in SEQ ID NO: 19.

SEQ ID NO: 52 is an amino and carboxy terminal addition of the amino acid sequence shown in SEQ ID NO: 19.

SEQ ID NO: 53 is an amino and carboxy terminal addition of the amino acid sequence shown in SEQ ID NO: 19.

### DETAILED DISCLOSURE OF THE INVENTION

One aspect of the subject invention concerns peptides having an amino acid sequence that comprises a cecropin A peptide sequence and a pleurocidin peptide sequence. In one embodiment, the peptide comprises a hinge region between the cecropin A sequence and the pleurocidin sequence. In a specific embodiment, the peptide comprises an N-terminal extension or a C-terminal extension or both an N-terminal extension and a C-terminal extension. In a further embodiment, the hinge region comprises a plurality of amino acids selected from the group consisting of G and I. In an exemplified embodiment, the hinge region comprises the amino acid sequence GIG. In one embodiment, the cecropin A sequence comprises an N-terminal sequence of cecropin A. In a specific embodiment, the cecropin A sequence comprises the amino acid sequence KWKLFKKI. In a specific embodiment, the modified pleurocidin sequence comprises the amino acid sequence FKKAAHVGKAAL. In one embodiment, a peptide of the invention comprises or consists of the amino acid sequence of SEQ ID NO: 2, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53. In a specific embodiment, the peptide comprises the amino acid sequence of SEQ ID NO: 2, or an amino acid sequence having at least 60% sequence identity with SEQ ID NO: 2, or a biologically active fragment or variant thereof.

A peptide of the invention can have the general formula of:

Xₙ₁—Y—Xₙ₂—Z—Xₙ₃

wherein

X=any amino acid

n1=0 to 7

n2=0 to 5

n3=0 to 7

Y=an amino acid sequence from the N-terminus region of cecropin A peptide.

Z=an amino acid sequence from pleurocidin.

In one embodiment, Y is the KWKFLKKI sequence from cecropin A, and/or Z is the sequence FKKAAHVGKAAL derived from pleurocidin. In one embodiment, Xₙ₂ comprises one or more G and/or I amino acids. In a specific embodiment, Xₙ₂ comprises the amino acid sequence GIG. In one embodiment, Xₙ₁ comprises one or more M and/or A amino acids. In one embodiment, Xₙ₃ comprises one or more T and/or K amino acids. In a specific embodiment, Xₙ₁ comprises the amino acid sequence MA. In a specific embodiment, Xₙ₃ comprises the amino acid sequence TK.

Peptides of the subject invention include the specific peptides exemplified herein as well as equivalent peptides which may be, for example, somewhat longer or shorter than the peptides exemplified herein. For example, using the teachings provided herein, a person skilled in the art could readily make peptides having from 1 to about 15 or more amino acids added to one or both ends of a peptide of the subject invention. Examples of peptides having amino acids added to one or both ends of the exemplified peptides (SEQ ID NO: 2 and SEQ ID NO. 19) and contemplated within the scope of the present invention are shown in SEQ ID NO: 24 to SEQ ID NO: 53. Similarly, a person skilled in the art could readily prepare peptides in which 1 to about 5 amino acids are removed from one or both ends of a peptide of the subject invention. Examples of peptide fragments of the exemplified peptides and contemplated within the scope of the present invention are shown in SEQ ID NO. 8 to SEQ ID NO. 23. The subject invention includes, but is not limited to, the exemplified longer and shorter peptides. Peptides wherein 1 to about 5 amino acids are added to one or both ends of the peptide WKLFKKIGIGFKKAAHVGKAA (SEQ ID NO: 2) or wherein 1 to about 5 amino acids are removed from one or both ends of the peptide are specifically exemplified below (wherein “X” in the sequence represents any amino acid):

(SEQ ID NO: 8)

AKWKLFKKIGIGFKKAAHVGKAALTK

(SEQ ID NO: 9)

KWKLFKKIGIGFKKAAHVGKAALTK

(SEQ ID NO: 10)

WKLFKKIGIGFKKAAHVGKAALTK

(SEQ ID NO: 11)

KLFKKIGIGFKKAAHVGKAALTK

(SEQ ID NO: 12)

LFKKIGIGFKKAAHVGKAALTK

(SEQ ID NO: 13)

MAKWKLFKKIGIGFKKAAHVGKAALT

(SEQ ID NO: 14)

MAKWKLFKKIGIGFKKAAHVGKAAL

(SEQ ID NO: 15)

MAKWKLFKKIGIGFKKAAHVGKAA

(SEQ ID NO: 16)

MAKWKLFKKIGIGFKKAAHVGKA

(SEQ ID NO: 17)

MAKWKLFKKIGIGFKKAAHVGK

(SEQ ID NO: 18)

AKWKLFKKIGIGFKKAAHVGKAALT

(SEQ ID NO: 19)

KWKLFKKIGIGFKKAAHVGKAAL

(SEQ ID NO: 20)

WKLFKKIGIGFKKAAHVGKAA

(SEQ ID NO: 21)

KLFKKIGIGFKKAAHVGKA

(SEQ ID NO: 22)

LFKKIGIGFKKAAHVGK

(SEQ ID NO: 23)

FKKIGIGFKKAAHVG

(SEQ ID NO: 24)

XMAKWKLFKKIGIGFKKAAHVGKAALTK

(SEQ ID NO: 25)

XXMAKWKLFKKIGIGFKKAAHVGKAALTK

(SEQ ID NO: 26)

XXXMAKWKLFKKIGIGFKKAAHVGKAALTK

(SEQ ID NO: 27)

XXXXMAKWKLFKKIGIGFKKAAHVGKAALTK

(SEQ ID NO: 28)

XXXXXMAKWKLFKKIGIGFKKAAHVGKAALTK

(SEQ ID NO: 29)

MAKWKLFKKIGIGFKKAAHVGKAALTKX

(SEQ ID NO: 30)

MAKWKLFKKIGIGFKKAAHVGKAALTKXX

(SEQ ID NO: 31)

MAKWKLFKKIGIGFKKAAHVGKAALTKXXX

(SEQ ID NO: 32)

MAKWKLFKKIGIGFKKAAHVGKAALTKXXXX

(SEQ ID NO: 33)

MAKWKLFKKIGIGFKKAAHVGKAALTKXXXXX

(SEQ ID NO: 34)

XMAKWKLFKKIGIGFKKAAHVGKAALTKX

(SEQ ID NO: 35)

XXMAKWKLFKKIGIGFKKAAHVGKAALTKXX

(SEQ ID NO: 36)

XXXMAKWKLFKKIGIGFKKAAHVGKAALTKXXX

(SEQ ID NO: 37)

XXXXMAKWKLFKKIGIGFKKAAHVGKAALTKXXXX

(SEQ ID NO: 38)

XXXXXMAKWKLFKKIGIGFKKAAHVGKAALTKXXXXX

(SEQ ID NO: 39)

XKWKLFKKIGIGFKKAAHVGKAAL

(SEQ ID NO: 40)

XXKWKLFKKIGIGFKKAAHVGKAAL

(SEQ ID NO: 41)

XXXKWKLFKKIGIGFKKAAHVGKAAL

(SEQ ID NO: 42)

XXXXKWKLFKKIGIGFKKAAHVGKAAL

(SEQ ID NO: 43)

XXXXXKWKLFKKIGIGFKKAAHVGKAAL

(SEQ ID NO: 44)

KWKLFKKIGIGFKKAAHVGKAALX

(SEQ ID NO: 45)

KWKLFKKIGIGFKKAAHVGKAALXX

(SEQ ID NO: 46)

KWKLFKKIGIGFKKAAHVGKAALXXX

(SEQ ID NO: 47)

KWKLFKKIGIGFKKAAHVGKAALXXXX

(SEQ ID NO: 48)

KWKLFKKIGIGFKKAAHVGKAALXXXXX

(SEQ ID NO: 49)

XKWKLFKKIGIGFKKAAHVGKAALX

(SEQ ID NO: 50)

XXKWKLFKKIGIGFKKAAHVGKAALXX

(SEQ ID NO: 51)

XXXKWKLFKKIGIGFKKAAHVGKAALXXX

(SEQ ID NO: 52)

XXXXKWKLFKKIGIGFKKAAHVGKAALXXXX

(SEQ ID NO: 53)

XXXXXKWKLFKKIGIGFKKAAHVGKAALXXXXX

Peptides included within the scope of the invention include peptides from about 15 to about 60 amino acids. Thus, within the scope of the invention are peptides of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 amino acids in length. In one embodiment, peptides of the invention consist of about 15 to about 40 amino acids. In another embodiment, peptides of the invention consist of about 20 to about 30 amino acids. All longer and shorter peptides are within the scope of the subject invention as long as the longer or shorter peptide retains substantially the same antimicrobial activity as the peptides exemplified herein. The subject invention also concerns polypeptides that comprise a peptide sequence of the present invention, or a fragment or variant of that sequence, and that exhibit antimicrobial activity.

The subject invention also concerns a polynucleotide comprising a nucleotide sequence encoding a peptide of the invention. In one embodiment, a polynucleotide of the invention encodes a peptide comprising or consisting of the amino acid sequence shown in any of SEQ ID NO: 2, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53, or a biologically active fragment or variant thereof. In a specific embodiment, a polynucleotide of the invention comprises the nucleotide sequence shown in SEQ ID NO: 1 or SEQ ID NO: 7, or a nucleotide sequence having 60% or greater sequence identity with SEQ ID NO: 1 or SEQ ID NO: 7, or a fragment or variant thereof.

The subject invention also concerns a method for providing a plant with resistance to a plant pathogen, said method comprising incorporating a polynucleotide of the invention into said plant.

The subject invention also concerns a cell comprising a polynucleotide of the invention or a peptide of any of the invention.

The subject invention also concerns a plant or plant tissue comprising a cell of the invention.

The subject invention also concerns a method for treating or preventing infection, or providing resistance to a pathogen, in a person or animal, said method comprising administering an effective amount of a peptide of the invention or a polynucleotide of the invention to said person or animal.

The subject invention also concerns a method for designing a polynucleotide sequence encoding a polypeptide exhibiting antimicrobial properties, said method comprising identifying a polynucleotide sequence encoding a polypeptide exhibiting antimicrobial activity; modifying said polynucleotide sequence so as to change physiochemical properties of said polypeptide encoded thereby to produce an optimized polypeptide, wherein said modifying comprises (i) increasing an average parameter value for α-helix conformation of said polypeptide, (ii) increasing hydrophobicity, amphipathicity, or hydrophilicity of said polypeptide, (iii) increasing net charge of said polypeptide, (iv) reducing disorder of said polypeptide, and/or (v) reducing potential protein interaction index of said polypeptide.

Another embodiment of the invention pertains to SEQ ID NO: 1 or a polynucleotide sequence that hybridizes to a compliment thereof under high stringency conditions (variant). In yet another embodiment, the subject invention pertains to a polypeptide having a amino acid sequence of SEQ ID NO: 2 or a polypeptide having at least 70 percent homology therwith.

The subject invention also pertains to a method of increasing resistance to pathogens, such as microbes, in a plant cell comprising transforming said plant cell with a vector comprising SEQ ID NO: 1. Another embodiment pertains to a plant cell transformed with SEQ ID NO: 1. Preferably, SEQ ID NO: 1 is introduced into such cell in such a way as to be expressed by the cell.

The subject invention also concerns a genetic construct or an expression construct comprising a plant promoter, a polynucleotide according to SEQ ID NO: 1 or SEQ ID NO: 7, or a fragment or variant thereof, and a termination sequence.

The subject invention also concerns a cell comprising a peptide of the invention, or a nucleic acid encoding a peptide of the invention. In on embodiment, the peptide comprises the amino acid sequence shown in SEQ ID NO: 2, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53, or a biologically active fragment or variant thereof. In a further embodiment, the nucleic acid comprises a polynucleotide sequence shown in SEQ ID NO: 1 or SEQ ID NO: 7, or a nucleotide sequence having 60% or greater sequence identity with SEQ ID NO: 1 or SEQ ID NO: 7. The cell can be a eukaryotic or prokaryotic cell. Preferably, the polynucleotide sequence is provided in an expression construct of the invention. The cell can be a prokaryotic cell, for example, a bacterial cell such as E. coli or B. subtilis, or the cell can be a eukaryotic cell, for example, a plant cell, including protoplasts, or an animal cell. Plant cells include, but are not limited to, dicotyledonous, monocotyledonous, and conifer cells. In one embodiment, the plant cell is a grape plant cell. In a specific embodiment, the plant cell is a cell from a V. vinifera plant. Animal cells include human cells, mammalian cells, avian cells, and insect cells. Mammalian cells include, but are not limited to, COS, 3T3, and CHO cells.

Single letter amino acid abbreviations are defined in Table 1.

TABLE 1

| Letter Symbol | Amino Acid |
|---|---|
| A | Alanine |
| B | Asparagine or |
| | aspartic acid |
| C | Cysteine |
| D | Aspartic Acid |
| E | Glutamic Acid |
| F | Phenylalanine |
| G | Glycine |
| H | Histidine |
| I | Isoleucine |
| K | Lysine |
| L | Leucine |
| M | Methionine |
| N | Asparagine |
| P | Proline |
| Q | Glutamine |
| R | Arginine |
| S | Serine |
| T | Threonine |
| V | Valine |
| W | Tryptophan |
| Y | Tyrosine |
| Z | Glutamine or |
| | glutamic acid |

Polynucleotides useful in the present invention can be provided in an expression construct. Expression constructs of the invention generally include regulatory elements that are functional in the intended host cell in which the expression construct is to be expressed. Thus, a person of ordinary skill in the art can select regulatory elements for use in bacterial host cells, yeast host cells, plant host cells, insect host cells, mammalian host cells, and human host cells. Regulatory elements include promoters, transcription termination sequences, translation termination sequences, enhancers, and polyadenylation elements. As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation.

An expression construct of the invention can comprise a promoter sequence operably linked to a polynucleotide sequence encoding a peptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site in the expression construct as it is from the transcription start site in its natural genetic environment. Some variation in this distance is permitted without substantial decrease in promoter activity. A transcription start site is typically included in the expression construct.

If the expression construct is to be provided in or introduced into a plant cell, then plant viral promoters, such as, for example, a cauliflower mosaic virus (CaMV) 35S (including the enhanced CaMV 35S promoter (see, for example U.S. Pat. No. 5,106,739)) or a CaMV 19S promoter or a cassava vein mosaic can be used. Other promoters that can be used for expression constructs in plants include, for example, prolifera promoter, Ap3 promoter, heat shock promoters, T-DNA 1′- or 2′-promoter of A. tumefaciens, polygalacturonase promoter, chalcone synthase A (CHS-A) promoter from petunia, tobacco PR-1a promoter, ubiquitin promoter, actin promoter, alcA gene promoter, pint promoter (Xu et al., 1993), maize WipI promoter, maize trpA gene promoter (U.S. Pat. No. 5,625,136), maize CDPK gene promoter, and RUBISCO SSU promoter (U.S. Pat. No. 5,034,322) can also be used. Tissue-specific promoters, for example fruit-specific promoters, such as the E8 promoter of tomato (accession number: AF515784; Good et al. (1994)) can be used. Fruit-specific promoters such as flower organ-specific promoters can be used with an expression construct of the present invention for expressing a polynucleotide of the invention in the flower organ of a plant. Examples of flower organ-specific promoters include any of the promoter sequences described in U.S. Pat. Nos. 6,462,185; 5,639,948; and 5,589,610. Seed-specific promoters such as the promoter from a grape 2S albumin gene (U.S. Pat. No. 7,250,296), β-phaseolin gene (for example, of kidney bean) or a glycinin gene (for example, of soybean), and others, can also be used. Endosperm-specific promoters include, but are not limited to, MEG1 (EPO application No. EP1528104) and those described by Wu et al. (1998), Furtado et al. (2001), and Hwang et al. (2002). Root-specific promoters, such as any of the promoter sequences described in U.S. Pat. No. 6,455,760 or U.S. Pat. No. 6,696,623, or in published U.S. patent application Nos. 20040078841; 20040067506; 20040019934; 20030177536; 20030084486; or 20040123349, can be used with an expression construct of the invention. Constitutive promoters (such as the CaMV, ubiquitin, actin, or NOS promoter), developmentally-regulated promoters, and inducible promoters (such as those promoters than can be induced by heat, light, hormones, or chemicals) are also contemplated for use with polynucleotide expression constructs of the invention.

Expression constructs of the invention may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides of the invention. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent. Examples include the maize shrunken-1 enhancer element (Clancy and Hannah, 2002).

DNA sequences which direct polyadenylation of mRNA transcribed from the expression construct can also be included in the expression construct, and include, but are not limited to, an octopine synthase or nopaline synthase signal. The expression constructs of the invention can also include a polynucleotide sequence that directs transposition of other genes, i.e., a transposon.

Polynucleotides of the present invention can be composed of either RNA or DNA. Preferably, the polynucleotides are composed of DNA. The subject invention also encompasses those polynucleotides that are complementary in sequence to the polynucleotides disclosed herein. Polynucleotides and polypeptides of the invention can be provided in purified or isolated form.

Because of the degeneracy of the genetic code, a variety of different polynucleotide sequences can encode peptides useful in the present invention. A table showing all possible triplet codons (and where U also stands for T) and the amino acid encoded by each codon is described in Lewin (1985). In addition, it is well within the skill of a person trained in the art to create alternative polynucleotide sequences encoding the same, or essentially the same, peptides of the subject invention. These variant or alternative polynucleotide sequences are within the scope of the subject invention. As used herein, references to “essentially the same” sequence refers to sequences which encode amino acid substitutions, deletions, additions, or insertions which do not materially alter the functional activity of the polypeptide encoded by the polynucleotides of the present invention. Allelic variants of the nucleotide sequences encoding a protein of the invention are also encompassed within the scope of the invention.

Substitution of amino acids other than those specifically exemplified or naturally present in a peptide of the invention are also contemplated within the scope of the present invention. For example, non-natural amino acids can be substituted for the amino acids of a peptide, so long as the peptide having the substituted amino acids retains substantially the same functional activity as the peptide in which amino acids have not been substituted. Examples of non-natural amino acids include, but are not limited to, ornithine, citrulline, hydroxyproline, homoserine, phenylglycine, taurine, iodotyrosine, 2,4-diaminobutyric acid, α-amino isobutyric acid, 4-aminobutyric acid, 2-amino butyric acid, γ-amino butyric acid, ε-amino hexanoic acid, 6-amino hexanoic acid, 2-amino isobutyric acid, 3-amino propionic acid, norleucine, norvaline, sarcosine, homocitrulline, cysteic acid, τ-butylglycine, τ-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form. Allelic variants of a protein sequence of the present invention are also encompassed within the scope of the invention.

Amino acids can be generally categorized in the following classes: non-polar, uncharged polar, basic, and acidic. Conservative substitutions whereby a peptide of the present invention having an amino acid of one class is replaced with another amino acid of the same class fall within the scope of the subject invention so long as the peptide having the substitution still retains substantially the same functional activity (e.g., antimicrobial activity) as the peptide that does not have the substitution. Polynucleotides encoding a peptide having one or more amino acid substitutions in the sequence are contemplated within the scope of the present invention. Table 2 below provides a listing of examples of amino acids belonging to each class.

TABLE 2

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

The subject invention also concerns variants of the polynucleotides of the present invention that encode biologically active peptides of the invention. Variant sequences include those sequences wherein one or more nucleotides of the sequence have been substituted, deleted, and/or inserted. The nucleotides that can be substituted for natural nucleotides of DNA have a base moiety that can include, but is not limited to, inosine, 5-fluorouracil, 5-bromouracil, hypoxanthine, 1-methylguanine, 5-methylcytosine, and tritylated bases. The sugar moiety of the nucleotide in a sequence can also be modified and includes, but is not limited to, arabinose, xylulose, and hexose. In addition, the adenine, cytosine, guanine, thymine, and uracil bases of the nucleotides can be modified with acetyl, methyl, and/or thio groups. Sequences containing nucleotide substitutions, deletions, and/or insertions can be prepared and tested using standard techniques known in the art.

Fragments and variants of a peptide of the present invention can be generated as described herein and tested for the presence of function using standard techniques known in the art. Thus, an ordinarily skilled artisan can readily prepare and test fragments and variants of a peptide of the invention and determine whether the fragment or variant retains functional activity relative to full-length or a non-variant peptide.

As well as the wild-type AMP polynucleotide or polypeptide sequences, alternative sequences having similarity may also be used. In the context of the present application, a polynucleotide sequence is “homologous” with the known sequence if at least 70%, preferably at least 80%, most preferably at least 90% of its base composition and base sequence corresponds to the reported naturally occurring sequence. According to the invention, a “homologous protein” is to be understood to comprise proteins which contain an amino acid sequence at least 70% of which, preferably at least 80% of which, most preferably at least 90% of which, corresponds to the amino acid of a given known amino acid sequence; wherein corresponds is to be understood to mean that the corresponding amino acids are either identical or are mutually homologous amino acids. The expression “homologous amino acids” denotes those which have corresponding properties, particularly with regard to their charge, hydrophobic character, steric properties, etc. Thus, in one embodiment the protein may be from 70% up to less than 100% homologous to an AMP.

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman (1981), to find the best segment of identity or similarity between two sequences. Gap performs global alignments: all of one sequence with all of another similar sequence using the method of Needleman and Wunsch (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Polynucleotides and polypeptides contemplated within the scope of the subject invention can also be defined in terms of more particular identity and/or similarity ranges with those sequences of the invention specifically exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and BLAST programs of Altschul et al., (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

The subject invention also contemplates those polynucleotide molecules having sequences which are sufficiently homologous with the polynucleotide sequences exemplified herein so as to permit hybridization with that sequence under standard stringent conditions and standard methods (Maniatis et al., 1982).

The terms “stringent conditions” or “stringent hybridization conditions” includes reference to conditions under which a polynucleotide will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA—DNA hybrids, the Tm can be approximated from the equation of Meinkoth and Wahl, Anal. Biochem., 138:267-284 (1984): Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with approximately 90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Current Protocols in Molecular Biology, Chapter 2, Ausubel, et al., Eds., Greene Publishing and Wiley-Interscience, New York (2000).

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the DNA strand that is transcribed. The polynucleotide sequences also include both full-length sequences as well as shorter sequences derived from the full-length sequences. Allelic variations of the exemplified sequences also fall within the scope of the subject invention. The polynucleotide sequence includes both the sense and antisense strands either as individual strands or in the duplex.

Plants within the scope of the present invention include monocotyledonous plants, such as, for example, rice, wheat, barley, oats, rye, sorghum, maize, sugarcane, pineapple, onion, bananas, coconut, lilies, turfgrasses, and millet. Plants within the scope of the present invention also include dicotyledonous plants, such as, for example, tomato, cucumber, squash, peas, alfalfa, melon, chickpea, chicory, clover, kale, lentil, soybean, beans, tobacco, potato, sweet potato, yams, cassaya, radish, broccoli, spinach, cabbage, rape, apple trees, citrus (including oranges, mandarins, grapefruit, lemons, limes and the like), grape, cotton, sunflower, strawberry, and lettuce. In one embodiment, the plant, plant tissue, or plant cell is tomato. In another embodiment, the plant, plant tissue, or plant cell is thale cress (Arabidopsis). Herb plants containing a polynucleotide of the invention are also contemplated within the scope of the invention. Herb plants include parsley, sage, rosemary, thyme, and the like. In a specific embodiment, the plant is a grape plant, such as Vitis vinifera.

Techniques for transforming plant cells with a gene are known in the art and include, for example, Agrobacterium infection, biolistic methods, electroporation, calcium chloride treatment, PEG-mediated transformation, etc. U.S. Pat. No. 5,661,017 teaches methods and materials for transforming an algal cell with a heterologous polynucleotide. Transformed cells can be selected, redifferentiated, and grown into plants that contain and express a polynucleotide of the invention using standard methods known in the art. The seeds and other plant tissue and progeny of any transformed or transgenic plant cells or plants of the invention are also included within the scope of the present invention.

Peptides of the invention, and fragments thereof, can be used to generate antibodies that bind specifically to a peptide of the invention, and such antibodies are contemplated within the scope of the invention. The antibodies of the invention can be polyclonal or monoclonal and can be produced and isolated using standard methods known in the art.

In one embodiment, one or more of the peptides of the subject invention can be provided in the form of a multiple peptide construct. Such a construct can be designed so that multiple peptides are linked to each other by intervening moieties wherein the intervening moieties are subsequently cleaved or removed following administration of the multiple peptide construct to a patient. Methods for constructing multiple peptide constructs are known in the art. For example, peptides of the present invention can be provided in the form of a multiple antigenic peptide (MAP) construct. The preparation of MAP constructs has been described in Tam (1988). MAP constructs utilize a core matrix of lysine residues onto which multiple copies of an immunogen are synthesized. Multiple MAP constructs, each containing different peptides, can be prepared and administered in accordance with methods of the present invention. In another embodiment, a multiple peptide construct can be prepared by preparing the subject peptides having at least one metal chelating amino acid incorporated therein, preferably at the amino and/or carboxy terminal of the peptide as described, for example, in U.S. Pat. No. 5,763,585. The peptides are then contacted with a solid support having attached thereto a metal ion specific for the metal chelating amino acid of the peptide. A multiple peptide construct of the invention can provide multiple copies of the exact same peptide, including variants or fragments of a subject peptide, or copies of different peptides of the subject invention.

Therapeutic application of the subject peptides, and compositions containing them, can be accomplished by any suitable therapeutic method and technique presently or prospectively known to those skilled in the art. The peptides can be administered by any suitable route known in the art including, for example, oral, nasal, rectal, parenteral, subcutaneous, or intravenous routes of administration. Administration of the peptides of the invention can be continuous or at distinct intervals as can be readily determined by a person skilled in the art.

Compounds and compositions useful in the subject invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the bioactive peptide is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art. Examples of carriers or diluents for use with the subject peptidomimetics include, but are not limited to, water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these. Formulations of the peptide, antibody, or peptidomimetic of the invention can also comprise suspension agents, protectants, lubricants, buffers, preservatives, and stabilizers. To provide for the administration of such dosages for the desired therapeutic treatment, pharmaceutical compositions of the invention will advantageously comprise between about 0.1% and 45%, and especially, 1 and 15% by weight of the total of one or more of the peptide, antibody, or peptidomimetic based on the weight of the total composition including carrier or diluent.

The compounds and molecules of the subject invention can also be administered utilizing liposome technology, slow release capsules, implantable pumps, and biodegradable containers. These delivery methods can, advantageously, provide a uniform dosage over an extended period of time.

The subject peptides can also be modified by the addition of chemical groups, such as PEG (polyethylene glycol). PEGylated peptides typically generate less of an immunogenic response and exhibit extended half-lives in vivo in comparison to peptides that are not PEGylated when administered in vivo. Methods for PEGylating proteins and peptides known in the art (see, for example, U.S. Pat. No. 4,179,337). The subject peptides can also be modified to improve cell membrane permeability. In one embodiment, cell membrane permeability can be improved by attaching a lipophilic moiety, such as a steroid, to the peptide. Other groups known in the art can be linked to peptides of the present invention.

The subject invention also concerns a packaged dosage formulation comprising in one or more containers at least one peptide, polynucleotide, or antibody of the subject invention formulated in a pharmaceutically acceptable dosage. The package can contain discrete quantities of the dosage formulation, such as tablet, capsules, lozenge, and powders. The quantity of peptide, polynucleotide, and/or antibody in a dosage formulation and that can be administered to a patient can vary from about 1 mg to about 2000 mg, more typically about 1 mg to about 500 mg, or about 5 mg to about 250 mg, or about 10 mg to about 100 mg.

The subject invention also concerns kits comprising in one or more containers a composition, compound, or peptide of the present invention. In one embodiment, a kit contains a peptide, polynucleotide, and/or antibody of the present invention. In a specific embodiment, a kit comprises a peptide having the amino acid sequence shown in SEQ ID NO. 2 or SEQ ID NO. 19, or a fragment or variant of the peptide. In a more specific embodiment, a kit comprises a peptide consisting of the amino acid sequence shown in SEQ ID NO. 2 or SEQ ID NO. 19.

The subject invention also concerns methods for preparing a peptide, polynucleotide or antibody of the invention. In one embodiment, a peptide or polynucleotide of the invention is chemically synthesized using standard methods. In another embodiment, a peptide or antibody of the invention is prepared by expressing a polynucleotide encoding the peptide or antibody either in vitro or in vivo and then isolating the expressed peptide or antibody.

Peptide design parameters. In order to use transgenic technology to control X. fastidiosa in grapevine while minimizing unintended health risks to consumers, a hybrid AMP was created by combining peptide sequences of cecropin A and pleurocidin. The exemplified hybrid peptide was named B-passerin based on the Latin names of the donor organisms (B-, an abbreviation for bombyx—the Latin name for silkworm and passer, the Latin name for flounder fish). Disclosed herein is the method of molecular design for active hybrid AMPs and the utilization of B-passerin to confer high levels of resistance to PD in transgenic grape plants.

The design of B-passerin was accomplished via the optimization of parameters for helical structure and peptide functionality using Vector NTI software and other DNA/protein analysis tools. B-passerin is composed of an N-terminal segment of cecropin A (residues 1-8) and a modified portion of pleurocidin (residues 6-14 plus 19-21) in an elaborate molecular framework consisting of N- and C-terminal extensions (MA to the N-terminus and TK to the C-terminus) and a hinge region (GIG). Sequence analyses revealed that the unique amino acid composition renders the B-passerin peptide an α-helix conformational profile superior to that of other hybrid AMPs including the CEMA-derived MsrA1. In addition, unlike CEMA that contains amino acid sequences from insect hosts, B-passerin is more amphipathic with a lower average hydrophobicity index, probably due to the water-adapted donor organism—flounder. In comparison with various well-studied AMPs, B-passerin shows several advantageous molecular characteristics essential for conferring effective antimicrobial activity. These include a high content of positively charged or basic residues (Lys) and non-polar hydrophobic residues (Ala), a high net positive charge, the absence of any negatively charged or hydrophilic residues, and a significantly lower index for potential protein interaction (PPI). PPI index has been used as an indicator for non-pore forming protein-protein interactive activities that might otherwise compromise both molecular stability and unintended side effects of the peptide molecules (Boman, 2003).

In planta test for antimicrobial activity. A polynucleotide sequence encoding the B-passerin peptide was synthesized based on grape codon preference. The B-passerin encoding sequence under the control of a bi-directional duplex promoter complex for enhanced constitutive expression was introduced into transgenic grape plants (Li et al., 2004). These plants were inoculated with X. fastidiosa bacterium and evaluated for their resistance to PD. Results indicated that B-passerin-expressing plants were able to survive stringent disease challenges with no or lessened visible symptoms for up to 12 months and produced morphological characteristics identical to non-transformed plants. Under the same stringent test conditions, all susceptible control plants developed severe PD symptoms and died within a time period of 8-10 weeks. These findings demonstrate that a B-passerin encoding sequence, when expressed constitutively in transgenic grape plants, is capable of conferring resistance to X. fastidiosa.

The newly developed B-passerin with demonstrated antimicrobial activity can provide sustainable PD resistance in otherwise susceptible V. vinifera grape cultivars and facilitate the advancement of grape production and the wine industry in PD-affected areas. It can also be utilized as an antimicrobial and therapeutic agent to provide resistance to pathogenic microorganisms in other plant species and animals.

### Structural Analysis of Cecropin Pleurocidin Hybrid Peptide B-Passerin

The B-passerin peptide was designed via the analysis and optimization of molecular parameters using Vector NTI DNA/protein analysis software. B-passerin peptide contains a 27-residue sequence: MAKWKLFKKIGIGFKKAAHVGKAALTK (SEQ ID NO: 2). Structurally, this peptide is composed of an N-terminal extension (MA) for optimal translation efficiency (Kozak, 1989), a basic region of cecropin A (residues 1-8) responsible for α-helix formation, a hinge segment (GIG), a modified portion of pleurocidin (residues 6-14 plus 19-21) and a basic extension (TK) to increase the overall cationicity. The B-passerin peptide without the terminal amino acid extensions is shown below:

| | |
|---|---|
| KWKLFKKIGIGFKKAAHVGKAAL. | (SEQ ID NO: 19) |

The structural organization of AMPs has been studied using techniques such as circular dichroism (Sitraram and Nagaraj, 1999). The inventors have realized that several potent linear AMPs, including cecropins, magainins, melittin and pleurocidin, are unordered in an aqueous environment but form an α-helix in structure-promoting solvents such as trifluoroethanol (TFE) and in lipid membrane environments (Sitraram and Nagaraj, 1999; Syvitski et al., 2005). Such structural alternations appear to be due to an intrinsic conformational propensity of AMPs, determined by amino acid sequence and ionic interactions of amino acid residues in response to different chemical environments. AMPs have a wide range of sequence compositions and thus vary significantly in ability to form α-helical structures and conformational stability.

The mechanisms of membrane permeabilization have been investigated using structure-to-function approaches with AMP molecules and various molecular techniques (Brogden, 2005). Using various models, a prerequisite for efficacious membrane spanning and permeation activity is the ability of AMP molecules to adopt and maintain an α-helical structure in the membrane environment. According to this structural concept, several hybrid AMPs and variants were designed and tested (Boman et al., 1989; Wade et al., 1990; Pier et al., 1994). However, these attempts to produce AMP hybrids relied mainly on a heuristic or empirical approach. Here we demonstrate the development of the amino acid sequence for the AMP hybrid B-passerin using parameters optimized via comparative theoretical analyses and computational tools. These include algorithmic conformational parameters for α-helix and hydrophobicity indices from Vector NTI DNA/protein analysis software (Deleage and Roux, 1987); GlobPlot for flexibility/disordered regions within peptide sequence (Linding et al., 2003); amino acid composition and net charge analyses from Vector NTI DNA/protein analysis software and potential protein interaction index via Molpep 3.5 (Boman, 2003).

Thus, according to one embodiment, the subject invention pertains to a method of optimizing a polynucleotide sequence encoding a polypeptide exhibiting antimicrobial properties so as to increase antimicrobial activity, said method comprising:

identifying a polynucleotide sequence encoding a polypeptide exhibiting antimicrobial activity;

modifying said polynucleotide sequence so as to change physiochemical properties of said polypeptide encoded thereby to produce an optimized polypeptide, wherein said modifying comprises

(i) increasing an average parameter value for α-helix conformation of said polypeptide,

(ii) increasing hydrophobicity, amphipathicity, or hydrophilicity of said polypeptide,

(iii) increasing net charge of said polypeptide,

(iv) reducing disorder of said polypeptide, and/or

(v) reducing potential protein interaction index of said polypeptide.

In a specific embodiment, the polynucleotide is modified by one, two, three, four or five of any of the foregoing modifying steps. According to another embodiment, the subject invention pertains to a polynucleotide produced by the foregoing method.

According to another embodiment, the subject invention pertains to a method of screening DNA sequences encoding polypeptides having enhanced antimicrobial potential, said method comprising screening a database containing genetic sequence information for sequences having at least a preselected identity to a polynucleotide sequence that encodes SEQ ID NO: 1.

The following SEQ ID NO: 1 is a nucleotide sequence (including a stop codon TGA at 3′ end) encoding a B-passerin peptide useful in accordance with the teachings herein. The peptide coding sequence only covers 81 by for 27 amino acid residues, while the following sequence is 84 by long.

(SEQ ID NO: 1)

atggctaagtggaagctcttcaagaagatcggcatcggtttcaagaaagc
cgcccatgtgggcaaggccgctctcaccaagtga.

Thus, the subject invention also concerns the following nucleotide sequence:

(SEQ ID NO: 7)

atggctaagtggaagctcttcaagaagatcggcatcggtttcaagaaagc
cgcccatgtgggcaaggccgctctcaccaag.

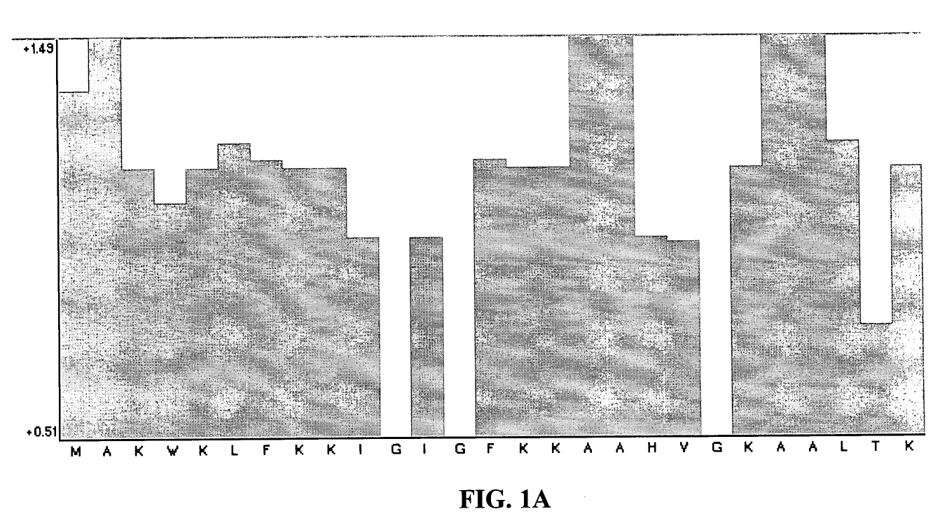

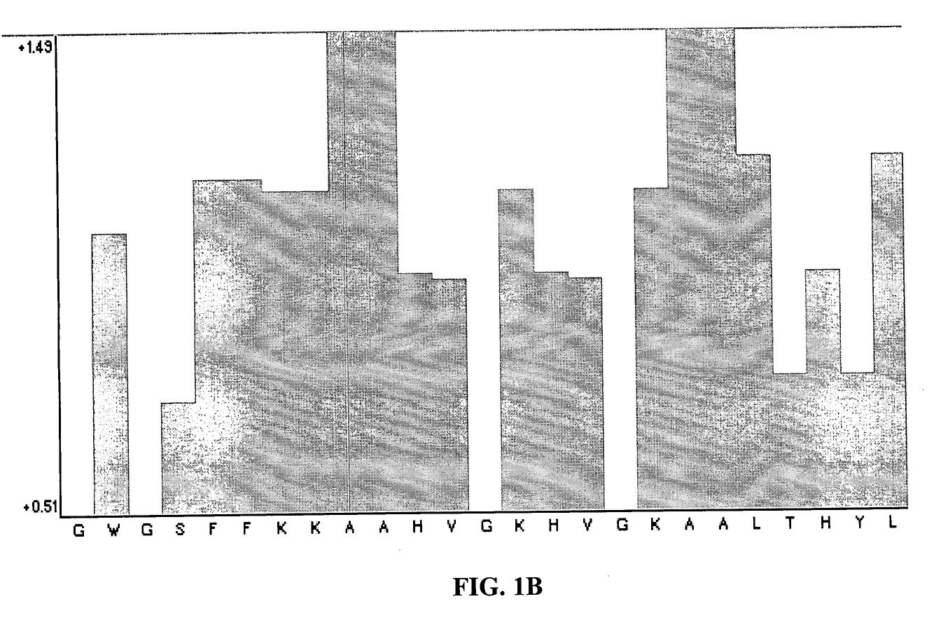

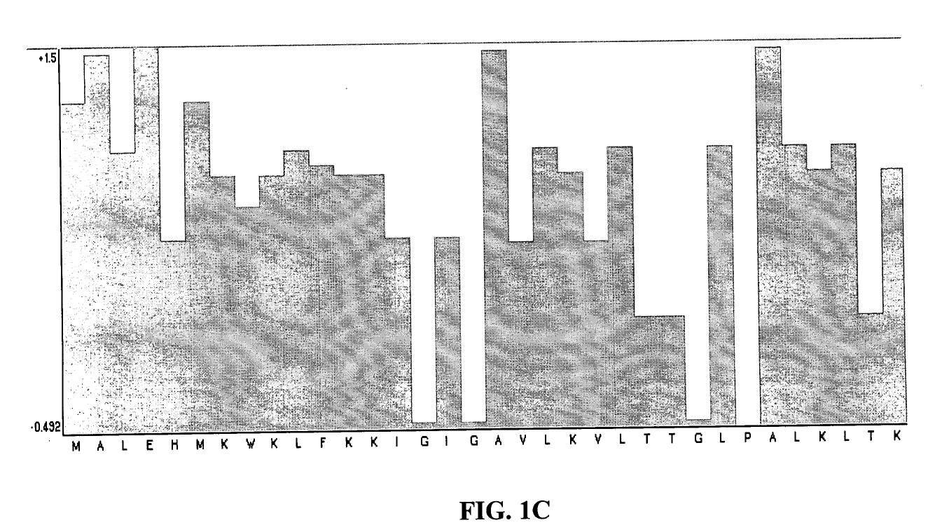

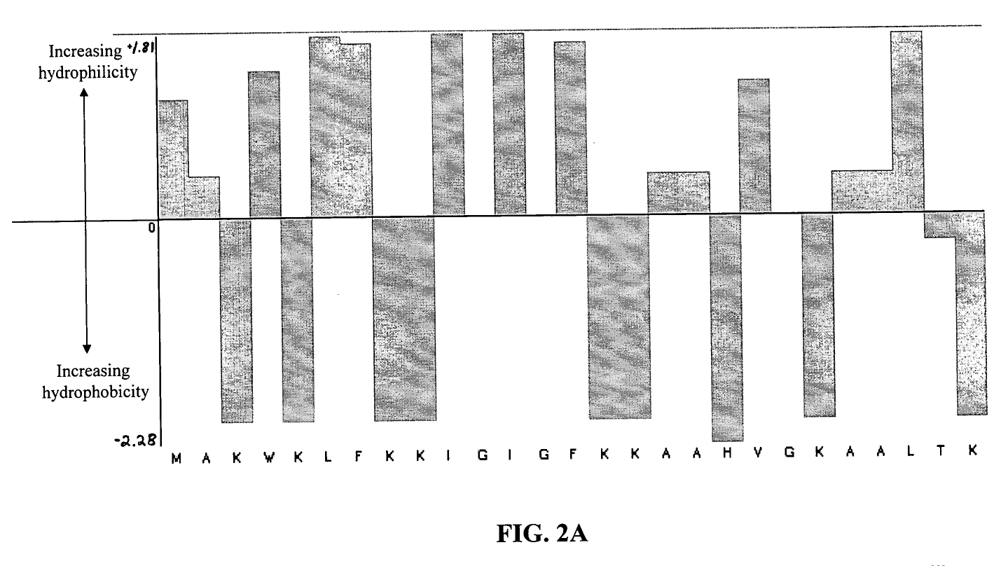

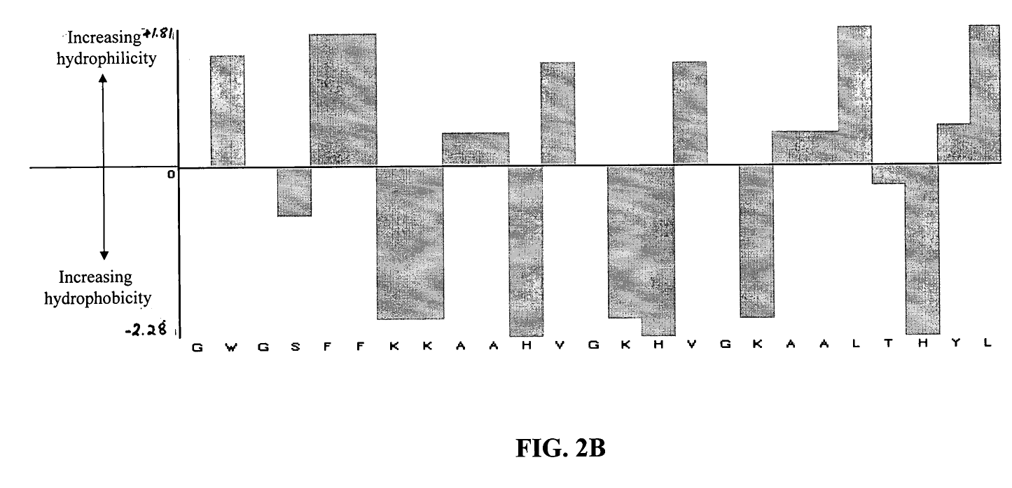

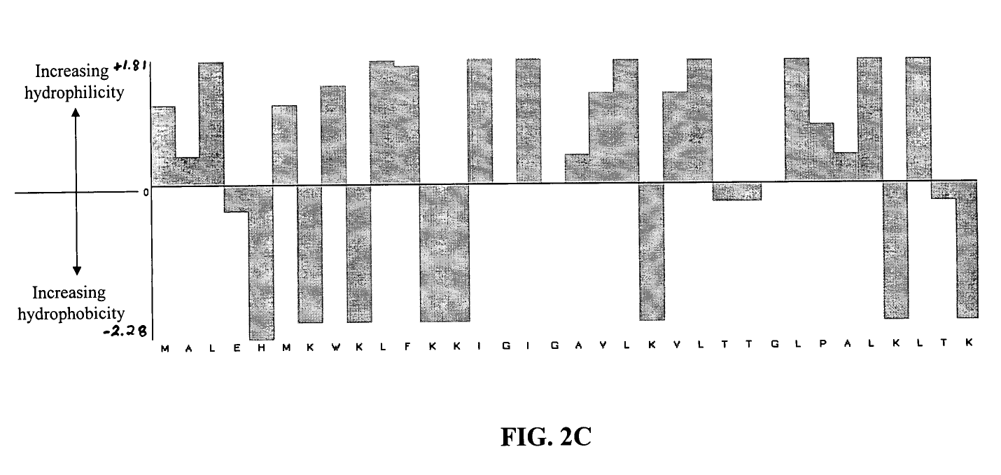

Three AMP peptides, B-passerin, pleurocidin and CEMA-derived Msr1A1, were compared for their α-helix conformational parameters using algorithmic predictions of their secondary structure (FIG. 1) (Deleage and Roux, 1987). Among these peptides, B-passerin showed a higher propensity to form an α-helical structure. It had the highest average parameter value for α-helix conformation (1.128) when compared to two other peptide molecules (pleurocidin=1.037, MsrA1=1.094). We postulate that these parameter values represent the tendency of peptides to become an α-helix in the membrane environment. B-passerin, with the highest value, may have the intrinsic capacity to form a more rigid α-helix than that of other AMP peptides. Similarly, comparison of hydrophobicity indices, determined by retention times of amino acid residues on reversed-phase high-performance liquid chromatography, revealed that B-passerin possesses an average hydrophobicity index value (−0.359) that is significantly lower than that of the other two AMP peptides (pleurocidin=−0.098; MsrA1=0.263) (FIG. 2). According to Cowan and Whittaker (1990), a lower hydrophobicity index value corresponds to increased hydrophobicity. Increased hydrophobicity of a lytic peptide is positively correlated with a favorable interaction with lipid bilayers and enhanced ability for spontaneous membrane insertion.

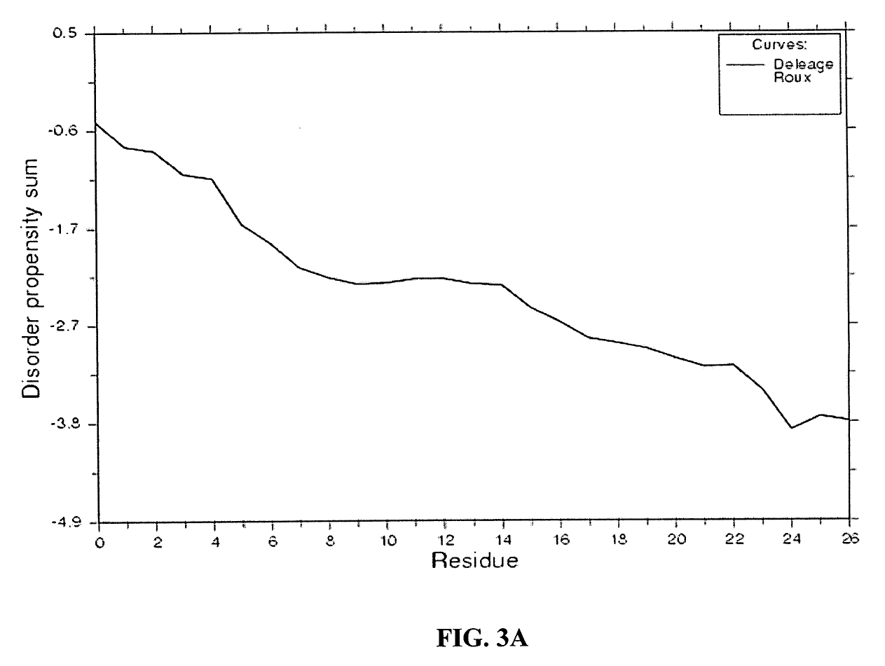

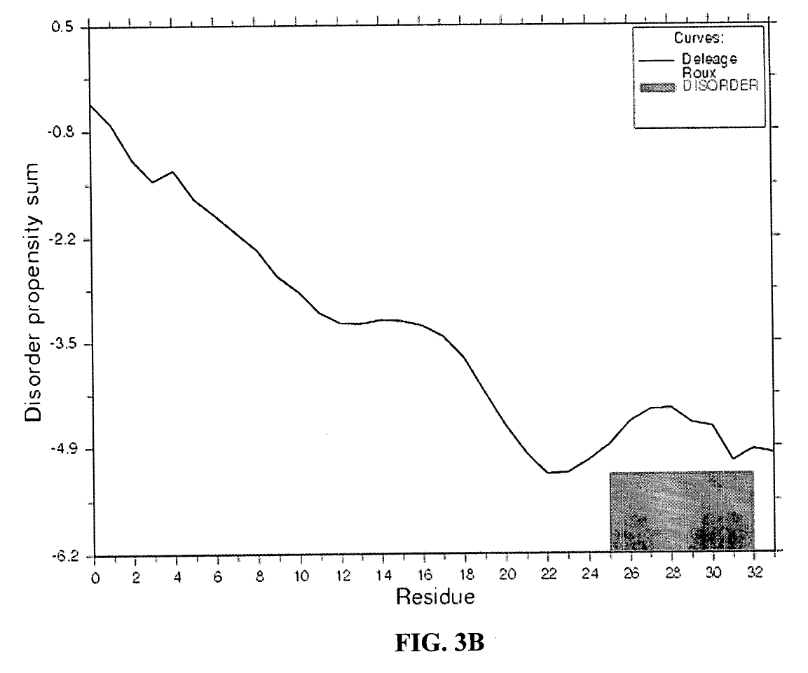

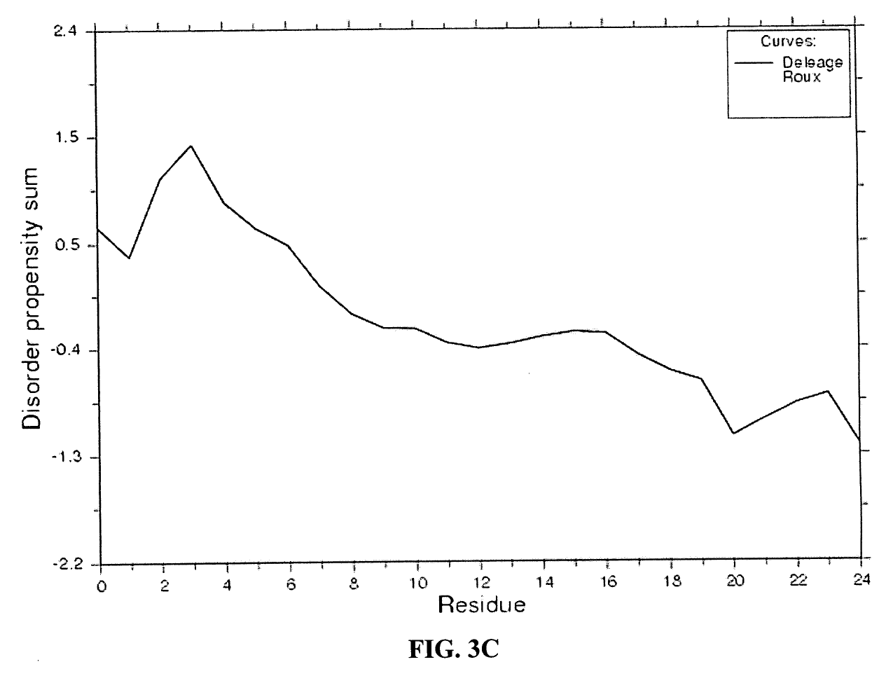

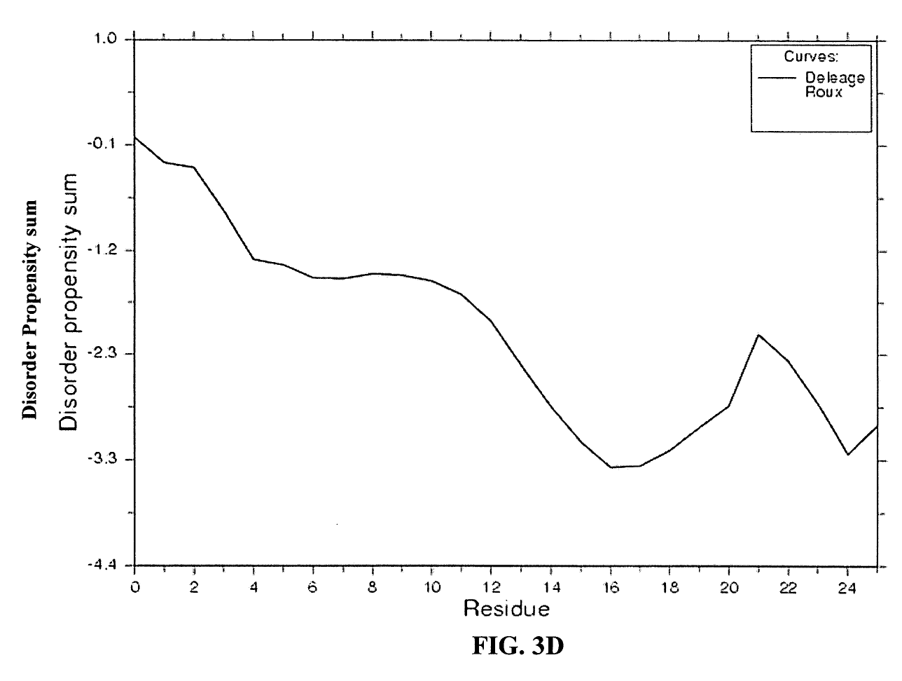

Analysis of several AMP peptides using GlobPlot and the Deleage/Roux definition for protein conformational propensity indicated that B-passerin has a propensity for amino acid residues to be in a relatively ordered state similar to that of the potent natural AMP pleurocidin. These data validate the assumption that B-passerin has a high tendency to form a rigid transmembrane helix as does pleurocidin (FIG. 3). On the other hand, based on our analyses, sequence modifications to the CEME hybrid peptide resulted in significantly altered structural organization and a highly disordered tail fragment in MsrA1 that might affect the helical structure of the peptide (FIG. 3).

Table 3 summarizes physiochemical properties of B-passerin and a number of AMPs. As discussed by Boman (2003), these features play an important role in the lytic action of the AMP against bacterial membranes and thus determine the overall functionality of the peptides. Among these AMPs, B-passerin has the highest net charge value and the highest percentage of positively charged or basic residues. A positive net charge is a critical indicator for the affinity of a lytic peptide to bind to bacterial phospholipids (Boman, 2003). In addition to a high percentage of basic residues, B-passerin also contains the highest percentage of lysine residues. Lysine residues contain a long and positively charged side chain. Many potent AMPs contain numerous lysine residues. These residues provide crucial structural and functional elements for the membrane spanning action of the lytic peptides.

Boman tested the use of a parameter called potential protein interaction index (PPI index) to distinguish bacterial membrane lytic activity from potentially unfavorable protein-protein interactions. This is an estimate of protein-binding potential for lytic peptides. The PPI index is determined by the sum of free energies from respective side chains released during transfer from cyclohexane to water (as determined by Radzeka and Wolfenden, 1988), divided by the total number of residues, minus proline (Boman, 2003). Based on Boman's analysis, several AMP peptides showed a high PPI index value. The predicted results were in agreement with observations that, besides having antimicrobial properties, these peptides tended to interact with other proteins in the host, acting as neurotransmitters and hormones. It was noted that all natural AMPs have positive PPI index values, while some synthetic hybrid AMPs, such as CEMA, have negative PPI index values. In our comparative analysis, B-passerin had the lowest PPI index value (0.20) among 9 peptides, excluding CEMA and its derivative MsrA1. The value is identical to that of pleurocidin. Unlike other marine lytic peptides, such as pardaxin that is secreted from mucous glands and acts as a neurotoxin, pleurocidin has no known harmful side effects or non-specific protein interactive activities against eukaryotic cells (Oren and Shai, 1996; Cole et al., 1997). Based on the similarity in the PPI index analysis, it is expected that pleurocidin-derived B-passerin should also provide optimal lytic activity against bacterial membrane without non-specific interactions with host membrane proteins.

The presence of alternating groups of 5 to 10 residues with high α-helical content separated by a few weak residues is an important factor for the structure-function relation of many AMPs and has been incorporated into the design of B-passerin. Yoshida et al., (2000) substituted two weak Gly residues with stronger Ala residues of pleurocidin to artificially enhance the α-helicity. As expected, the increased overall α-helical content in the hydrophobic region of pleurocidin resulted from such Gly-Ala substitutions enhanced the antibacterial activity. However, the same substitution modification seemed to abolish the alternating pattern of α-helicity of pleurocidin and resulted in a dramatic increase in hemolysis, turning pleurocidin, which has little hemolytic activity, into an effective hemolytic agent (Yoshida et al., 2000).

### Construction of a Gene Encoding Lytic Peptide B-Passerin and Transformation Vectors

In one embodiment, a B-passerin peptide of the invention contains 27 amino acid residues (MAKWKLFKKIG IGFKKAAHVGKAALTK) (SEQ ID NO: 2). A 84-bp double stranded DNA sequence (having a stop codon at the 3′ terminus) coding for the B-passerin peptide was synthesized by ligation of chemically synthesized oligonucleotide primers (atggctaagt ggaagctctt caagaagatc ggcatcggtt tcaagaaagc cgcccatgtg ggcaaggccg ctctcaccaa gtga) (SEQ ID NO: 1). In designing the DNA sequences, codons preferably used by V. vinifera species were chosen to encode each amino acid residue at the DNA level (see codon usage table for V. vinifera from http://www.kazusa.or.jp/codon/cgi-bin/showcodon.cgi?species=Vitis+vinifera+[gbpin]). The final gene sequence was subsequently cloned into a pUC-19 plasmid vector and nucleotide sequences were confirmed by DNA sequencing.

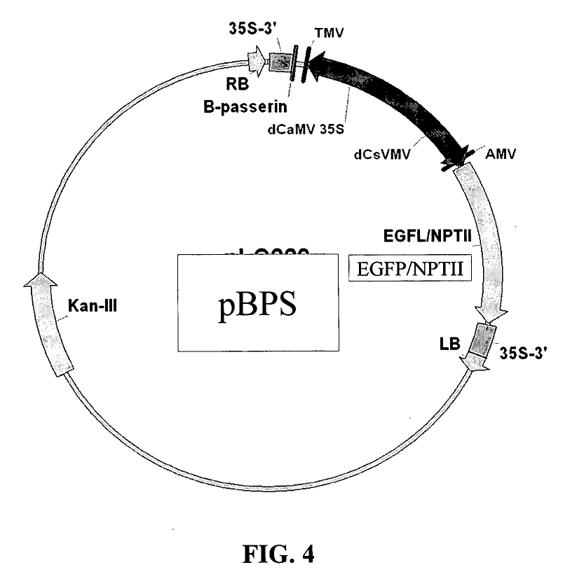

The B-passerin gene was further subcloned into an expression cassette under control of a constitutive double-enhanced CaMV 35S promoter and subsequently introduced into a binary vector pDCsVM that contained a bi-functional fusion EGFP/NPT-II marker gene expression unit (Li et al., 2001), resulting in transformation vector pBPS (FIG. 4). In pBPS, the lytic peptide gene expression unit was placed in a divergent orientation with the double enhanced promoter complex that controls the expression of the fusion marker gene, thus forming a bi-directional duplex promoter complex (BDPC) for the constitutive expression of both peptide and fusion marker genes. BDPC has been shown to be capable of conferring a significantly enhanced level of gene expression for associated transgenes in transgenic plants (Li et al., 2004).

pBPS and other control binary vectors, including pCM that contain a CEMA-derived MsrA1 gene in a similar configuration of gene expression units, were introduced into Agrobacterium tumefaciens strain EHA105 and then used in subsequent transformation experiments.

### Agrobacterium-Mediated Transformation and Recovery of Transgenic Grape Plants

Transgenic grape plants of cv. Thompson Seedless expressing the B-passerin gene were obtained after Agrobacterium-mediated transformation of grape SE using a previously described procedure with modifications (Li et al., 2001). Thompson Seedless was used due to the high transformation efficiencies that we obtain routinely in our laboratory. Thompson Seedless is, as are all other V. vinifera cultivars, highly susceptible to PD. Thus, it provides a suitable test subject for transgene-induced PD resistance. All transgenic plants showed normal transgene expression based on the visualization of GFP-specific fluorescence derived from the expression of the EGFP-NPT-II fusion marker gene (Li et al., 2001).

Confirmation of Resistance to Xylella Fastidiosa from Lytic Peptide Chimeras Via Greenhouse Inoculation Test

Transgenic and control plants were grown in the greenhouse for about one to two months to reach a plant height of about 3 to 4 feet. Plants were then inoculated by injecting two drops (30 μl each) of pathogenic X. fastidiosa bacterial suspension into xylem tissue at the base of the main stems. The bacterial inoculum was prepared by diluting an overnight bacterial culture to an OD₆₀₀ value of 0.2 or a titer of 1×10⁷ cfu/ml prior to use. Plants were continuously maintained in the greenhouse under normal growth conditions. About six weeks after inoculation, plants were evaluated for the development of PD symptoms including “marginal burning” on affected leaves (Hopkins and Purcell, 2002), followed by defoliation and eventual cessation of growth. A PD resistance index with a scale from 0 to 5 (susceptible to highly resistant) was used to quantify the resistance performance of tested plants. Based on this index, a plant that died from PD received a score of 0, whereas score numbers 1 to 5 were given to plants showing progressively lessened severity of PD symptoms and increasing plant vigor. A symptomless plant with robust PD resistance acquired a score of 5.

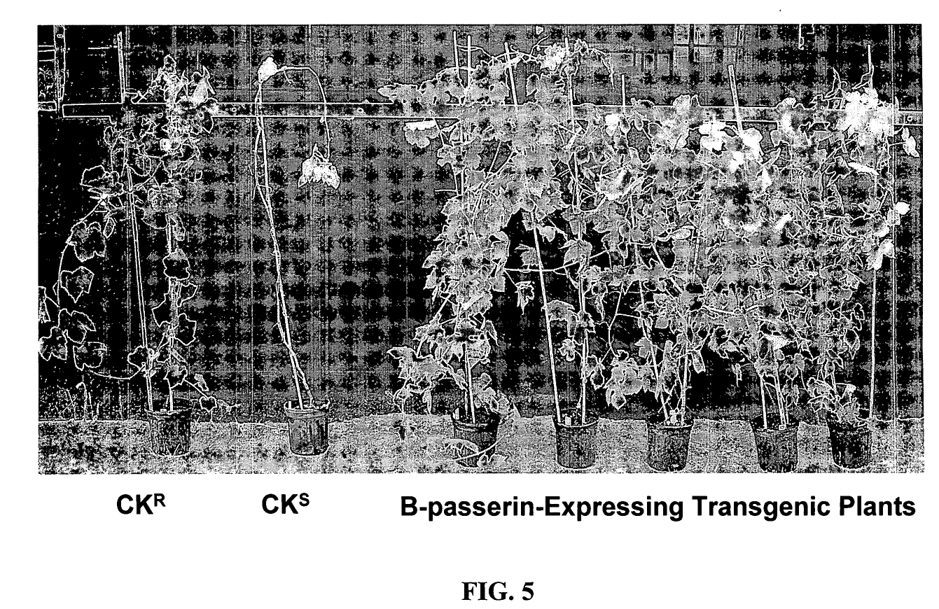

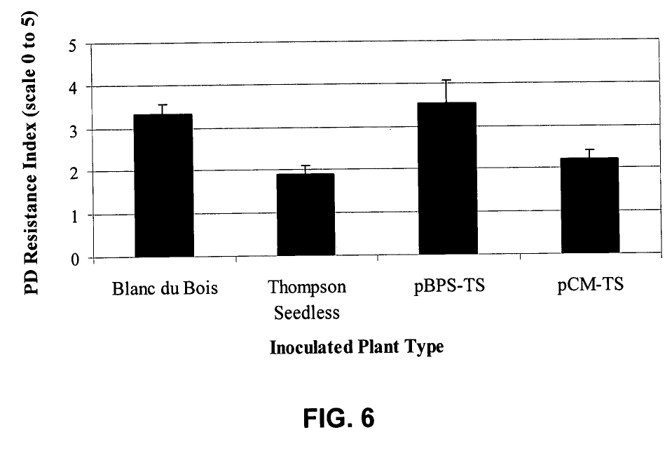

Since early June of 2005, 5 inoculation experiments have been conducted to test various independent transgenic plant lines expressing the B-passerin and other AMP genes, including the MsrA1 gene, and control plants. The average PD resistance indicies obtained 6 weeks after inoculation from different groups of inoculated plants are presented in FIG. 6. In these experiments, all susceptible control plants produced typical PD symptoms with correspondingly low resistance index values. These plants died, as judged by complete defoliation and cessation of growth, within a time period of 8-10 weeks post-inoculation due to severe clogging of xylem system by X. fastidiosa (FIG. 5, control plant CK^{s}). Furthermore, all resistant controls, including Florida hybrid bunch grape Blanc du Bois and rootstock Tampa, also developed pronounced PD symptoms and some eventually died due to severe disease development (FIG. 5, control plant CK^{R}). It should be noted that these local varieties are considered tolerant to PD and rarely develop symptoms in the field. The development of PD in such resistant control plants indicates the high stringency of our test conditions.

The majority of B-passerin-expressing plants (pBPS-TS) produced either no symptoms or reduced PD symptoms, resulting in PD resistance index values higher than that of tolerant control plants (FIG. 6). Thus far, 90% of the B-passerin expressing plants have survived the high stringency greenhouse test, while only about 60% of the control transgenic plants expressing other hybrid AMP genes including the MsrA1 gene (pCM-TS) survived. All surviving B-passerin expressing plants have remained symptomless for over 8 months and continue to grow vigorously (FIG. 5). These results indicate the correlation between expression of the B-passerin gene and the impediment of bacterial propagation and colonization within the xylem of these transgenic plants.

Known and/or yet to be discovered antimicrobial peptides (AMPs) are utilized as discussed herein. Exemplary AMPs which can, in toto or in part, be used in accordance with the methods of designing polypeptides optimized for conferring resistance include, but are not limited to, AMPs disclosed in Zasloff (2002); Boman (2003); and Tossi et al. (2000). The foregoing references provide examples of antimicrobial peptides with sequence information.

### Significance

Natural antimicrobial peptides have been isolated from numerous organisms, ranging from bacteria to animals, since 1970' s. Recent advancements in molecular technologies allow routine cloning and trans-expression of foreign genes in various transgenic target hosts. However, the use of genes encoding natural lytic AMPs, including cecropins, to confer resistance to phytopathogens in transgenic plants has thus far proven relatively ineffective and has failed to produce practical levels of resistance to phytopathogenic bacteria (Hightower et al., 1994; Florack et al., 1995; Hancock and Lehrer, 1998).

In an effort to improve the antimicrobial activity of AMPs in target hosts, several hybrid peptides composed of basic α-helical regions of cecropin A and melittin were chemically synthesized and evaluated for biological activity (Boman et al., 1989; Wade et al., 1990). Based on a heuristic approach for peptide sequence construction and screening, a hybrid peptide chimera CA-(1-8)-M-(1-18)NH2 was found to possess a level of antibacterial activity up to 100 times higher than that of cecropin A, magainin or melittin. This chimera also had an erthrocyte lysis value up to 3 times lower than that of cecropin A (Wade et al., 1990). It is important to note that cecropins are well known for their inability to lyse eukaryotic cells in spite of their high potency against a wide spectrum of bacteria, thus suggesting that they will simultaneously provide pathogen resistance and little-to-no human toxicity.

Following up on these findings, other research groups worldwide tested a variety of similar hybrid peptides and analogues with various degrees of modification at the amino acid sequence level. Subsequently, the use of several genes coding for these modified AMPs, analogues or hybrids was shown to provide limited resistance to disease in transgenic target hosts (Boman et al., 1989; Owens and Heutte, 1997; Arce et al., 1999; Osusky et al., 2000; Scorza and Gray, 2001 (U.S. Pat. No. 6,232,528)).

The previously reported MsrA1 gene was introduced into transgenic grape plants to serve as a positive control in experiments (Osusky et al., 2000). However, the MsrA1 gene was found relatively ineffective in providing resistance to the xylem-limited bacterium X. fastidiosa. All transgenic plants expressing the MsrA1 gene eventually succumbed to the high disease pressure in our greenhouse tests, except for a single plant line that showed reduced PD symptoms. The inability of this gene to confer resistance to X. fastidiosa in transgenic grape may be attributable to the reduced level of antibacterial activity associated with the use of hexapeptide extension (Osusky et al., 2000) and the instability of the peptide molecule within the aqueous xylem environment where X. fastidiosa resides. We also speculate that the inconsistent resistance performance may be caused by the incorporation of several destabilizing amino acid residues including L, E and H within the N-terminal extension (Varshaysky, 1996) and the addition of a C-terminal extension that substantially increased the disorder propensity of the peptide molecule in aqueous environment as revealed by GlobPlot (FIG. 3).

The subject application concerns a B-passerin gene encoding a cecropin-pleurocidin hybrid. It has been demonstrated that this peptide chimera is capable of conferring a significantly higher level of resistance to X. fastidiosa in transgenic grape plants. It should be emphasized that previous studies revealed that natural pleurocidin only conferred an antimicrobial activity against pathogenic microorganisms at moderate levels that were 10 to 20 times lower than that of CEMA. No amino acid sequences identical to that of B-passerin have been tested previously. The analytical method utilized to design hybrid peptide molecules, such as B-passerin, for improved functionality based on molecular and physiochemical characteristics also is novel.

### Notable Disadvantages of Conventional Practice Overcome by the Present Invention

Over the years, many natural AMPs isolated from various donor organisms have been utilized whole or in part for the development of resistance to phytopathogens. Results of their use as antimicrobial agents and therapeutics have been mixed. In many cases, the AMPs that resulted in high levels of resistance in transgenic plants were venomous in nature. Health risks associated with these peptides cannot be ignored. These compounds often function as potent allergens or even as neurotoxins capable of triggering adverse and, in some cases, fatal responses in humans. For instance, melittin is a well-known bee venom toxin that is capable of inducing an allergic reaction in humans with symptoms that range from extreme pain to anaphylactic shock. Victims often need to have immediate medical attention and treatments that range from administration of antihistamine or epinephrine to hospitalization. In addition, the use of venomous AMPs as ingredients in medicines or as food supplements is subject to strict regulatory approval to ensure consumer safety.

Many hybrid AMPs have been extensively studied for their cytotoxic effects using in vitro assays and animal models (e.g. Osusky et al., 2000). However, the safety of these compounds when consumed in large quantities in transgenic plants has not been evaluated. In addition, skepticism remains about the consumer acceptance of transgenic crops containing molecular components derived from well-known venomous peptides.

Pleurocidin has a broad spectrum of antimicrobial activity against Gram-positive and Gram-negative pathogenic bacteria and fungi. It has the typical molecular characteristic of lytic peptide, i.e., a state of an unordered molecule but assuming a rigid α-helical structure in a lipid membrane environment essential for spanning bacterial membranes. However, the level of antimicrobial activity of pleurocidin remains moderate. The molecular sequence of pleurocidin has been modified and tested for enhanced antimicrobial activity (U.S. Pat. Nos. 6,288,212 and 6,818,407). These studies showed the use of tested pleurocidin hybrids/analogs has been unsuccessful mainly due to the fact that all attempted peptide modifications connote either the loss of lytic activity or an increase in hemolytic activity (Patrzykat et al. 2002; Hancock et al., 2001, U.S. Pat. No. 6,288,212, and U.S. Pat. No. 6,818,407). The use of hybrid AMPs containing pleurocidin sequence components in transgenic plants to confer resistance to phytopathogens has not been reported.

A pleurocidin-derived hybrid peptide of the invention, B-passerin, simultaneously exhibits high bacterial membrane lytic activity and peptide stability in planta. Transgenic grapevine plants that express B-passerin exhibit resistance to xylem-limited phytopathogenic bacterium X. fastidiosa at levels significantly higher than that from other existing strong lytic AMPs.

Pleurocidin is well-known for its non-venomous nature and has no known toxic effect on eukaryotic cells. The absence of cytoxicity of pleurocidin is attributed to its unique amino acid composition associated with a low level of protein-protein interaction. Recently, in an effort to reduce the cytotoxicity of venomous peptide melittin, Asthana et al. (2004) discovered that heptadic leucine residues were responsible for the formation of a leucine zipper motif that resulted in cytotoxic activity. The substitution of leucine residues with hydrophobic alanine residues in this motif resulted in a dramatic reduction of the hemolytic activity of melittin. Based on our analysis, pleurocidin contains the highest percent of alanine residues (16%) among natural AMPs (Table 3). The B-passerin disclosed herein is further enhanced for its alanine content (17.86%). Based on these molecular characteristics, we postulate that B-passerin is non-toxic to eukaryotic cells and is capable of providing both safe and effective antimicrobial activity for human use.

The demonstrated method of designing lytic peptides with enhanced functionality based on molecular and physiochemical characteristics will have broad application for development of other AMP products. Further, B-passerin and similarly-derived peptides that exhibit such high levels of antimicrobial activity and low cytotoxicity will have potential as antimicrobial agents in other areas of application.

All patents, patent applications, provisional applications, and publications referred to or cited herein are incorporated by reference in their entirety, including all figures and tables, to the extent they are not inconsistent with the explicit teachings of this specification.

TABLE 3
Physicochemical properties of selected AMPs

| | | Net | % | % K | % A | PPI |
|---|---|---|---|---|---|---|
| Peptide | Residues | charge | Basic | (Lys) | (Ala) | Index* |
| B-passerin | 27 | −7.84 | 29.63 | 29.63 | 17.86 | 0.20 |
| Cecropin B | 35 | −6.76 | 25.71 | 20.00 | 14.29 | 0.88 |
| Shiva-1a | 38 | −6.76 | 23.68 | 5.26 | 12.82 | 2.12 |
| CEMA | 28 | −6.75 | 25.00 | 25.00 | 7.14 | −0.47 |
| MsrA1 | 34 | −5.84 | 20.59 | 20.59 | 8.57 | −0.38 |
| Cecropin A | 37 | −5.76 | 21.62 | 18.92 | 13.51 | 0.85 |
| MSI-99 | 24 | −4.76 | 25.00 | 25.00 | 8.33 | 0.50 |
| Pleurocidin | 25 | −4.02 | 16.00 | 16.00 | 16.00 | 0.20 |
| Magainin 2 | 23 | −2.85 | 17.39 | 17.39 | 8.70 | 0.42 |

*Potential Protein Interaction Index: Boman 2003/Molpep 3.5 at www.ki.se/jim

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 5,593,866
U.S. Pat. No. 5,707,855
U.S. Pat. No. 6,288,212
U.S. Pat. No. 6,818,407
U.S. Pat. No. 6,232,528
Arce P, Moreno M, Gutierrez M, Gebauer M, Dell'Orto P, Torres H, Acuna I, Oliger P, Venegas A, Jordana X, Kalazich J and Holuigue L (1999) “Enhanced resistance to bacterial infection by Erwinia carotovora subsp. atroseptica in transgenic potato plants expressing the attacin or the cecropin SB-37 genes” American Journal of Potato Research 76: 169-177.
Asthana N, Yadav S P and Ghosh J K (2004) “Dissection of antibacterial and toxic activity of melittin” J Biol Chem 279:55042-55050.
Boman H G (2003) “Antibacterial peptides: basic facts and emerging concepts” J Intern Med. 254:197-215.
Boman H G and Hultmark D (1987) “Cell-free immunity in insects' Annu Rev Microbiol 41:103-26.
Boman H G, Wade D, Boman I A, Wahlin B and Merrifield (1989) “Antibacterial and antimalarial properties of peptides that are cecropin-melittin hybrids” FEBS Lett 259:103-106.
Brogden K A (2005) “Antimicrobial peptides: pore formers or metabolic inhibitors in bacteria?” Nature Reviews 3:238-250.
Burrowes O J, Hadjicharalambous C, Diamond G and Lee T C (2004) “Evaluation of antimicrobial spectrum and cytotoxic activity of pleurocidin for food application” J Food Sci 69:N77-82.
Christensen B, Fink J, Merrifield R B and Mauzerall D (1988) “Channel-forming properties of cecropins and related model compounds incorporated into planar lipid membranes” PNAS 85:5072-5076.
Cole A M, Weis P and Diamond G (1997) “Isolation and characterization of pleurocidin, an antimicrobial peptide in the skin secretions of winter flounder” J Biol Chem 272:12008-12013.
Cole A M, Darouiche R O, Legarda D, Connell N and Diamond G (2000) “Characterization of fish antimicrobial peptide: gene expression, subcellular localization, and spectrum of activity” Antimicrob Agents & Chemother 44:2039-2045.
Deleage G and Roux B (1987) “An algorithm for protein secondary structure prediction based on class prediction” Protein Engin 1:289-294.
Ehrenstein G and Lecar H (1977) “Electrically gated ionic channels in lipid bilayers” Q Rev Biophys 10:1-34.
Florack D, Allefs S, Bollen R, Basch D, Visser B and Stiekema W (1995) “Expression of giant silkmoth cecropin B genes in tobacco” Transgenic Res 4:132-141.
Friedrich C, Scott M G, Karunaratne N, Yan H and Hancock R E W (1999) “Salt-resistant alpha-helical cationic antimicrobial peptides” Antimicrob Agents Chemother 43:1542-1548.
Friedrich C L, Moyles D, Beveridge T J and Hancock R E W (2000) “Antibacterial action of structurally diverse cationic peptides on Gram-positive bacteria” Antimicro Agents and Chemoth 44:2068-2092.
Gazit A, Boman A, Boman H and Shai Y (1992) “Interaction of the mammalian antibacterial peptide cecropin P1 with phospholipid vesicles” Biochem 34:11479-11488.
Hanbermann E (1972) “Bee and wasp venoms” Science 177:314-22.
Hancock R E W and Lehrer R (1998) “Cationic peptides: a new source of antibiotics” Trend in Biotechnol 16:82-88.
Hightower R, Baden C, Penzes E and Dunsmuir P (1994) “The expression of cecropin peptide in transgenic tobacco does not confer resistance to Pseudomonas syringae pv tabaci” Plant Cell Rep 13:295-299.
Hopkins D L and Purcell A H (2002) “Xylella fastidiosa: cause of Pierce's disease of grapevine and other emergent diseases” Plant Disease 86:1056-1066.
Huang Y and McBeath J H (1997) “Plant promoters controlled expression of cecropin B gene in transgenic potatoes renders disease resistance to soft rot bacteria” Phytopathology 87:S45. (Abstr.).
Jia X, Patrzykat A, Devlin R H, Ackerman P A, Iwama G K and Hancock R E W (2000) “Antimicrobial peptides protect coho salmon from Vibrio anguillarum infections” Appl Environm Microbiol 66:1928-1932.
Kozak M (1989) “Scanning model for translation: an update”. J Cell Biol 108:229-241.
Lee J Y, Boman A, Sun C, Andersson M, Jornvall H, Mutt V and Boman H G (1989) “Antibacterial peptides from pig intestine: isolation of a mammalian cecropin” PNAS 86:9159-9162.
Li Z T, Jayasankar S and Gray D J (2001) “Expression of a bifunctional green fluoresenct protein (GFP) fusion marker under the control of three constitutive promoters and enhanced derivatives in transgenic grape (Vitis vinifera)” Plant Sci 160:877-887.
Li Z T, Jayasankar S and Gray D J (2004) “Bi-directional duplex promoters with duplicated enhancers significantly increase transgene expression in grape and tobacco” Transgenic Res 13:143-154.
Linding R, Russell R B, Neduva V and Gibson T J (2003) “GlobPlot: exploring protein sequences for globularity and disorder” NAR 31:3701-3708.
Matsuzaki K, Murase O, Fujii N and Miyajima K (1996) “An antimicrobial peptide, magainin 2, induced rapid flip-flop of phospholipids coupled with pore formation and peptide translocation” Biochem 35:11361-11368.
Needleman and Wunsch (1970) J. Mol. Biol. 48:443-453.
Norelli J L, Borejsza-Wysocka E, Momol M T, Mills J Z, Grethel A, Alkwinckle H S, Ko K, Brown S K, Bauer D W, Beer S V, Abdul-Kader A M and Hanke V (1999) “Genetic transformation for fire blight resistance in apple” Acta Horticulturae 489:295-296.
Oren Z and Shai Y (1996) “A class of highly potent antibacterial peptides derived from pardaxin, a pore-forming peptide isolated from Moses sole fish Pardachirus marmoratus” Eur J Biochem 237:303-310.
Osusky M, Zhou G, Osuska L, Hancock R E W, Kay W W and Misra S (2000) “Transgenic plants expressing cationic peptide chimeras exhibit broad-spectrum resistance to phytopathogens” Nature Biotechn 18:1162-1166.
Owens L D and Heutte T M (1997) “A single amino acid substitution in the antimicrobial defense protein cecropin B is associated with diminished degradation by leaf intercellular fluid” Mol Plant-Microbe Inter 10:525-528.
Patrzykat A, Friedrich C L, Zhang L, Mendoza V and Hancock R E W (2002) “Sublethal concentrations of pleurocidin-derived antimicrobial peptides inhibit macromolecular synthesis in Escherichia coli” Antimicrob Agents Chemother 46:605-614.
Piers K L, Brown M H and Hancock R E W (1994) “Improvement of outer membrane-permeabilizing and lipopolysaccharide-binding activities of an antimicrobial cationic peptide by C-terminal modification” Antimicrob Agents Chemother 38:2311-2316.
Pouny Y and Shai Y (1992) ‘Interaction of D-amino acid incorporated analogues of pardaxin with membranes” Biochem 31:9482-9490.
Radzeka A and Wolfenden R (1988) “Comparing the polarities of amino acids: side-chain distribution coefficients between vapor phase, cyclohexane, 1-octanol and neutral aqueous solution” Biochem 27:1664-1670.
Sitaram N and Nagaraj R (1999) “Interaction of antimicrobial peptides with biological amd model membranes: structure and charge requirements for activity” Biochem Biophys Acta 1462:29-54.
Smith and Waterman (1981) Advances in Applied Mathematics 2: 482-489.
Steiner H, Hultmark D, Engstrom A, Bennich H, Boman H G (1981) “Sequence and specificity of two antibacterial proteins involved in insect immunity” Nature 292:246-8.
Syvitski R T, Burton I, Mattatall N R, Douglas S E and Jakeman D L (2005) “Structural characterization of the antimicrobial peptide pleurocidin from winter flounder” Biochem 44:7282-7293.
Tossi et al. (2000) “Amphipathic, alpha-helical Antimicrobial Peptides”, Biopolymers, 55:4-30.
Van Hofsten P, Faye I, Kockum K, Lee J-Y, Xanthopoulos K G, Boman I A, Boman H G, Engstrom A, Andreu D and Merrifield R B (1985) “Molecular cloning, cDNA sequencing, and chemical synthesis of cecropin B from Hyalophora cecropia” PNAS 1985 82: 2240-2243.
Varshaysky A (1996) ‘The N-end rule: functions, mysteries, uses” PNAS 93:12142-12149.
Wade D, Boman A, Wahlin B, Drain C M, Andreu D, Boman H G and Merrifield R B (1990) “All-D amino acid-containing channel-forming antibiotic peptides” PNAS 87:4761-4765.
Yang L, Harroun T A Weiss T M, Ding L and Huang H W (2001) “Barrel-stave model or toroidal model? A case study in mellitin pores” Biophys J 81:1475-1485.
Zasloff (2002) “Antimicrobial peptides of multicellular organisms”, Nature, 415:389-95.
Zhang L, Rozek A and Hancock R E W (2001) “Interaction of cationic antimicrobial peptides with model membranes” J Biol Chem 276:35714-35722.

1. A peptide comprising an amino acid sequence of a cecropin A peptide and a modified amino acid sequence of a pleurocidin peptide.

2. The peptide according to claim 1, wherein said peptide comprises a hinge region between said cecropin A sequence and said pleurocidin sequence.

3. The peptide according to claim 1, wherein said peptide comprises an N-terminal extension or a C-terminal extension or both an N-terminal extension and a C-terminal extension.

4. The peptide according to claim 2, wherein said hinge region comprises a plurality of amino acids selected from the group consisting of G and I.

5. The peptide according to claim 4, wherein said hinge region comprises the amino acid sequence GIG.

6. The peptide according to claim 1, wherein said cecropin A sequence comprises an N-terminal sequence of cecropin A.

7. The peptide according to claim 1, wherein said cecropin A sequence comprises the amino acid sequence KWKLFKKI.

8. The peptide according to claim 1, wherein said modified pleurocidin sequence comprises the amino acid sequence FKKAAHVGKAAL.

9. The peptide according to claim 1, wherein said peptide comprises the amino acid sequence of SEQ ID NO: 2, or an amino acid sequence having at least 60% sequence identity with SEQ ID NO: 2, or a biologically active fragment thereof.

10. A polynucleotide comprising a nucleotide sequence encoding a peptide comprising an amino acid sequence of a cecropin A peptide and a modified amino acid sequence of a pleurocidin peptide.

11. The polynucleotide according to claim 10, wherein said polynucleotide comprises the nucleotide sequence of SEQ ID NO: 1 or SEQ ID NO: 7.

12. A method for providing a plant with resistance to a plant pathogen, said method comprising incorporating a polynucleotide into said plant, wherein said polynucleotide comprises a nucleotide sequence encoding a peptide comprising an amino acid sequence of a cecropin A peptide and a modified amino acid sequence of a pleurocidin peptide.

13. The method according to claim 12, wherein said plant is a grape plant.

14. The method according to claim 13, wherein said grape plant is Vitis vinifera.

15. A cell comprising a polynucleotide comprising a nucleotide sequence encoding a peptide comprising an amino acid sequence of a cecropin A peptide and a modified amino acid sequence of a pleurocidin peptide, or comprising a peptide comprising an amino acid sequence of a cecropin A peptide and a modified amino acid sequence of a pleurocidin peptide.

16. A plant or plant tissue comprising a cell, wherein said cell comprises a polynucleotide comprising a nucleotide sequence encoding a peptide comprising an amino acid sequence of a cecropin A peptide and a modified amino acid sequence of a pleurocidin peptide, or said cell comprises a peptide comprising an amino acid sequence of a cecropin A peptide and a modified amino acid sequence of a pleurocidin peptide.

17. A method for treating or preventing infection, or providing resistance to a pathogen, in a person or animal, said method comprising administering an effective amount of a peptide comprising an amino acid sequence of a cecropin A peptide and a modified amino acid sequence of a pleurocidin peptide, or a polynucleotide comprising a nucleotide sequence encoding a peptide comprising an amino acid sequence of a cecropin A peptide and a modified amino acid sequence of a pleurocidin peptide to said person or animal.

18. (canceled)

19. The peptide according to claim 1, wherein said peptide comprises an amino acid sequence of SEQ ID NO: 2, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53, or a biologically active fragment thereof; or said peptide consists of an amino acid sequence of SEQ ID NO: 2, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31. SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53, or a biologically active fragment thereof.

20. (canceled)

21. The polynucleotide according to claim 10, wherein said polynucleotide encodes a peptide comprising the amino acid sequence of SEQ ID NO:2, or an amino acid sequence having at least 60% sequence identity with SEQ ID NO: 2, or a biologically active fragment thereof.

22. The method according to claim 12, wherein said polynucleotide encodes a peptide comprising the amino acid sequence of SEQ ID NO:2, or an amino acid sequence having at least 60% sequence identity with SEQ ID NO: 2, or a biologically active fragment thereof.

23. The method according to claim 12, wherein said polynucleotide comprises the nucleotide sequence of SEQ ID NO:1 or SEQ ID NO:7.

24. The method according to claim 17, wherein said peptide comprises the amino acid sequence of SEQ ID NO: 2, or an amino acid sequence having at least 60% sequence identity with SEQ ID NO: 2, or a biologically active fragment thereof.

25. The method according to claim 17, wherein said polynucleotide comprises the nucleotide sequence of SEQ ID NO:1 or SEQ ID NO:7.


SEQUENCE LISTING (53 sequences)

SEQ ID NO: 1 - DNA, 84 bp, artificial sequence
  a polynucleotide sequence encoding a B-passerin peptide of the invention.
atggctaagtggaagctcttcaagaagatcggcatcggtttcaagaaagccgcccatgtgggcaaggccgctctcaccaagtga

SEQ ID NO: 2 - protein, 27 aa, artificial sequence
  an amino acid sequence of a B-passerin peptide of the invention.
MAKWKLFKKIGIGFKKAAHVGKAALTK

SEQ ID NO: 3 - protein, 26 aa, artificial sequence
  amino acid sequence of a hybrid AMP designated as CEME.
KWKLFKKIGIGAVLKVLTTGLPALIS

SEQ ID NO: 4 - protein, 27 aa, artificial sequence
  amino acid sequence of a hybrid AMP designated as CEMA
KWKLFKKIGIGAVLKVTTGLPALKTLK

SEQ ID NO: 5 - protein, 6 aa, artificial sequence
  amino acid sequence of a peptide extension to the CEMA peptide.
MALEHM

SEQ ID NO: 6 - protein, 34 aa, artificial sequence
  the amino acid sequence of a hybrid AMP designated as MsrA1.
MALEHMKWKLFKKIGIGAVLKVLTTGLPALKTLK

SEQ ID NO: 7 - DNA, 81 bp, artificial sequence
  a polynucleotide sequence encoding a B-passerin peptide of the invention.
atggctaagtggaagctcttcaagaagatcggcatcggtttcaagaaagccgcccatgtgggcaaggccgctctcaccaag

SEQ ID NO: 8 - protein, 26 aa, artificial sequence
  an amino terminal deletion of the amino acid sequence shown in SEQ ID NO 2.
AKWKLFKKIGIGFKKAAHVGKAALTK

SEQ ID NO: 9 - protein, 25 aa, artificial sequence
  an amino terminal deletion of the amino acid sequence shown in SEQ ID NO 2.
KWKLFKKIGIGFKKAAHVGKAALTK

SEQ ID NO: 10 - protein, 24 aa, artificial sequence
  an amino terminal deletion of the amino acid sequence shown in SEQ ID NO 2.
WKLFKKIGIGFKKAAHVGKAALTK

SEQ ID NO: 11 - protein, 23 aa, artificial sequence
  an amino terminal deletion of the amino acid sequence shown in SEQ ID NO 2.
KLFKKIGIGFKKAAHVGKAALTK

SEQ ID NO: 12 - protein, 22 aa, artificial sequence
  an amino terminal deletion of the amino acid sequence shown in SEQ ID NO 2.
LFKKIGIGFKKAAHVGKAALTK

SEQ ID NO: 13 - protein, 26 aa, artificial sequence
  a carboxy terminal deletion of the amino acid sequence shown in SEQ ID NO 2.
MAKWKLFKKIGIGFKKAAHVGKAALT

SEQ ID NO: 14 - protein, 25 aa, artificial sequence
  a carboxy terminal deletion of the amino acid sequence shown in SEQ ID NO 2.
MAKWKLFKKIGIGFKKAAHVGKAAL

SEQ ID NO: 15 - protein, 24 aa, artificial sequence
  a carboxy terminal deletion of the amino acid sequence shown in SEQ ID NO 2.
MAKWKLFKKIGIGFKKAAHVGKAA

SEQ ID NO: 16 - protein, 23 aa, artificial sequence
  a carboxy terminal deletion of the amino acid sequence shown in SEQ ID NO 2.
MAKWKLFKKIGIGFKKAAHVGKA

SEQ ID NO: 17 - protein, 22 aa, artificial sequence
  a carboxy terminal deletion of the amino acid sequence shown in SEQ ID NO 2.
MAKWKLFKKIGIGFKKAAHVGK

SEQ ID NO: 18 - protein, 25 aa, artificial sequence
  an amino and carboxy terminal deletion of the amino acid sequence shown in SEQ ID NO 2.
AKWKLFKKIGIGFKKAAHVGKAALT

SEQ ID NO: 19 - protein, 23 aa, artificial sequence
  an amino and carboxy terminal deletion of the amino acid sequence shown in SEQ ID NO 2.
KWKLFKKIGIGFKKAAHVGKAAL

SEQ ID NO: 20 - protein, 21 aa, artificial sequence
  an amino and carboxy terminal deletion of the amino acid sequence shown in SEQ ID NO 2.
WKLFKKIGIGFKKAAHVGKAA

SEQ ID NO: 21 - protein, 19 aa, artificial sequence
  an amino and carboxy terminal deletion of the amino acid sequence shown in SEQ ID NO 2.
KLFKKIGIGFKKAAHVGKA

SEQ ID NO: 22 - protein, 17 aa, artificial sequence
  an amino and carboxy terminal deletion of the amino acid sequence shown in SEQ ID NO 2.
LFKKIGIGFKKAAHVGK

SEQ ID NO: 23 - protein, 15 aa, artificial sequence
  an amino and carboxy terminal deletion of the amino acid sequence shown in SEQ ID NO 2.
FKKIGIGFKKAAHVG

SEQ ID NO: 24 - protein, 28 aa, artificial sequence
  an amino terminal addition of the amino acid sequence shown in SEQ ID NO 2.
XMAKWKLFKKIGIGFKKAAHVGKAALTK

SEQ ID NO: 25 - protein, 29 aa, artificial sequence
  an amino terminal addition of the amino acid sequence shown in SEQ ID NO 2.
XXMAKWKLFKKIGIGFKKAAHVGKAALTK

SEQ ID NO: 26 - protein, 30 aa, artificial sequence
  an amino terminal addition of the amino acid sequence shown in SEQ ID NO 2.
XXXMAKWKLFKKIGIGFKKAAHVGKAALTK

SEQ ID NO: 27 - protein, 31 aa, artificial sequence
  an amino terminal addition of the amino acid sequence shown in SEQ ID NO 2.
XXXXMAKWKLFKKIGIGFKKAAHVGKAALTK

SEQ ID NO: 28 - protein, 32 aa, artificial sequence
  an amino terminal addition of the amino acid sequence shown in SEQ ID NO 2.
XXXXXMAKWKLFKKIGIGFKKAAHVGKAALTK

SEQ ID NO: 29 - protein, 28 aa, artificial sequence
  a carboxy terminal addition of the amino acid sequence shown in SEQ ID NO 2.
MAKWKLFKKIGIGFKKAAHVGKAALTKX

SEQ ID NO: 30 - protein, 29 aa, artificial sequence
  a carboxy terminal addition of the amino acid sequence shown in SEQ ID NO 2.
MAKWKLFKKIGIGFKKAAHVGKAALTKXX

SEQ ID NO: 31 - protein, 30 aa, artificial sequence
  a carboxy terminal addition of the amino acid sequence shown in SEQ ID NO 2.
MAKWKLFKKIGIGFKKAAHVGKAALTKXXX

SEQ ID NO: 32 - protein, 31 aa, artificial sequence
  a carboxy terminal addition of the amino acid sequence shown in SEQ ID NO 2.
MAKWKLFKKIGIGFKKAAHVGKAALTKXXXX

SEQ ID NO: 33 - protein, 31 aa, artificial sequence; identical to SEQ ID NO: 32
  a carboxy terminal addition of the amino acid sequence shown in SEQ ID NO 2.

SEQ ID NO: 34 - protein, 29 aa, artificial sequence
  an amino and carboxy terminal addition of the amino acid sequence shown in SEQ ID NO 2.
XMAKWKLFKKIGIGFKKAAHVGKAALTKX

SEQ ID NO: 35 - protein, 31 aa, artificial sequence
  an amino and carboxy terminal addition of the amino acid sequence shown in SEQ ID NO 2.
XXMAKWKLFKKIGIGFKKAAHVGKAALTKXX

SEQ ID NO: 36 - protein, 33 aa, artificial sequence
  an amino and carboxy terminal addition of the amino acid sequence shown in SEQ ID NO 2.
XXXMAKWKLFKKIGIGFKKAAHVGKAALTKXXX

SEQ ID NO: 37 - protein, 35 aa, artificial sequence
  an amino and carboxy terminal addition of the amino acid sequence shown in SEQ ID NO 2.
XXXXMAKWKLFKKIGIGFKKAAHVGKAALTKXXXX

SEQ ID NO: 38 - protein, 37 aa, artificial sequence
  an amino and carboxy terminal addition of the amino acid sequence shown in SEQ ID NO 2.
XXXXXMAKWKLFKKIGIGFKKAAHVGKAALTKXXXXX

SEQ ID NO: 39 - protein, 24 aa, artificial sequence
  an amino terminal addition of the amino acid sequence shown in SEQ ID NO 19.
XKWKLFKKIGIGFKKAAHVGKAAL

SEQ ID NO: 40 - protein, 25 aa, artificial sequence
  an amino terminal addition of the amino acid sequence shown in SEQ ID NO 19.
XXKWKLFKKIGIGFKKAAHVGKAAL

SEQ ID NO: 41 - protein, 26 aa, artificial sequence
  an amino terminal addition of the amino acid sequence shown in SEQ ID NO 19.
XXXKWKLFKKIGIGFKKAAHVGKAAL

SEQ ID NO: 42 - protein, 27 aa, artificial sequence
  an amino terminal addition of the amino acid sequence shown in SEQ ID NO 19.
XXXXKWKLFKKIGIGFKKAAHVGKAAL

SEQ ID NO: 43 - protein, 28 aa, artificial sequence
  an amino terminal addition of the amino acid sequence shown in SEQ ID NO 19.
XXXXXKWKLFKKIGIGFKKAAHVGKAAL

SEQ ID NO: 44 - protein, 24 aa, artificial sequence
  a carboxy terminal addition of the amino acid sequence shown in SEQ ID NO 19.
KWKLFKKIGIGFKKAAHVGKAALX

SEQ ID NO: 45 - protein, 25 aa, artificial sequence
  a carboxy terminal addition of the amino acid sequence shown in SEQ ID NO 19.
KWKLFKKIGIGFKKAAHVGKAALXX

SEQ ID NO: 46 - protein, 26 aa, artificial sequence
  a carboxy terminal addition of the amino acid sequence shown in SEQ ID NO 19.
KWKLFKKIGIGFKKAAHVGKAALXXX

SEQ ID NO: 47 - protein, 26 aa, artificial sequence
  a carboxy terminal addition of the amino acid sequence shown in SEQ ID NO 19.
KWKLFKKIGIGFKKAAHVGKAALXXX

SEQ ID NO: 48 - protein, 28 aa, artificial sequence
  a carboxy terminal addition of the amino acid sequence shown in SEQ ID NO 19.
KWKLFKKIGIGFKKAAHVGKAALXXXXX

SEQ ID NO: 49 - protein, 25 aa, artificial sequence
  an amino and carboxy terminal addition of the amino acid sequence shown in SEQ ID NO 19.
XKWKLFKKIGIGFKKAAHVGKAALX

SEQ ID NO: 50 - protein, 27 aa, artificial sequence
  an amino and carboxy terminal addition of the amino acid sequence shown in SEQ ID NO 19.
XXKWKLFKKIGIGFKKAAHVGKAALXX

SEQ ID NO: 51 - protein, 29 aa, artificial sequence
  an amino and carboxy terminal addition of the amino acid sequence shown in SEQ ID NO 19.
XXXKWKLFKKIGIGFKKAAHVGKAALXXX

SEQ ID NO: 52 - protein, 31 aa, artificial sequence
  an amino and carboxy terminal addition of the amino acid sequence shown in SEQ ID NO 19.
XXXXKWKLFKKIGIGFKKAAHVGKAALXXXX

SEQ ID NO: 53 - protein, 33 aa, artificial sequence
  an amino and carboxy terminal addition of the amino acid sequence shown in SEQ ID NO 19.
XXXXXKWKLFKKIGIGFKKAAHVGKAALXXXXX